(19) United States
(12) Patent Application Publication
(10) Pub. No.: US 2007/0270392 A1
(43) Pub. Date: Nov. 22, 2007

(54) Use of a Cyclopentenone Prostaglandin for Delaying the Onset and/or Preventing the Continuation of Labour

(75) Inventors: Phillip Robert Bennett, London (GB); Tamsim Lindstrom, London (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Imperial College Innovations Limited, London (GB)

(21) Appl. No.: 10/581,389

(22) PCT Filed: Mar. 29, 2004
(86) PCT No.: PCT/GB04/01380

§ 371 Date: Jun. 20, 2007

(30) Foreign Application Priority Data
Dec. 2, 2003 (GB) 0327975.9

(51) Int. Cl. A61K 31/5575; A61K 31/133; A61K 31/40; A61P 29/00; A61P 43/00; A61P 31/00; A61K 31/56; A61K 31/435

(52) U.S. Cl. 514/171; 514/356; 514/422; 514/573; 514/653

(57) ABSTRACT

The present invention provides the use of a cyclopentenone prostaglandin in the manufacture of a medicament for delaying the onset and/or preventing the continuation of labour in a female. Preferably the cyclopentenone prostaglandin prevents and/or reduces an inflammatory response in the reproductive system of a female. Preferably, the cyclopentenone prostaglandin is 15-deoxy-Δ^{12,14}-prostaglandin J₂ or prostaglandin A₁, or a precursor thereof. The invention further provides a pharmaceutical composition comprising cyclopentenone prostaglandin and methods of use thereof.

The present invention relates to agents for improving perinatal outcome in pre-term labour. In particular, the present invention relates to the use of prostaglandins to prevent and/or reduce an inflammatory response in the reproductive system of a female, thereby delaying the onset of labour.

Human pre-term labour, defined as spontaneous labour occurring prior to 37 weeks of gestation (with 39 weeks being term) continues to be a major problem, particularly in developed countries. Preterm birth occurs in 5-10% of all pregnancies but is associated with 70% of all neonatal deaths and up to 75% of neonatal morbidity (Rush et al., 1976). Premature neonates are at high risk of cerebral palsy, developmental delay, visual and hearing impairment and chronic lung disease.

During pregnancy, the uterus is maintained in a state of non-contractile quiescence whilst the cervix remains firm and closed. With the onset of labour, the cervix needs to become softer and to offer low resistance to force applied and have fibres which move under tension. The uterus also needs to begin contracting.

Both at term and preterm, the biochemistry of labour resembles an inflammatory reaction and there is accumulating evidence pointing to a pivotal role for pro-inflammatory cytokines and prostaglandins (PGs) in the labour process. Interleukin-1β (IL-1β) levels are elevated in amniotic fluid (Romero et al., 1990), gestational membranes (Keelan et al., 1999; Elliot et al., 2001) and the lower uterine segment (Maul et al., 2002) at term labour, and may contribute to labour onset by stimulating IL-8 and PG synthesis (Mitchell et al., 1990; Brown et al., 1998). PGs increase in maternal urine and blood and in fetal membranes in association with labour (Satoh et al., 1979; Skinner and Challis, 1985). PGE₂ stimulates uterine contractions (Dyal and Crankshaw, 1985), indirectly increases fundamentally dominant myometrial contractility by upregulation of oxytocin receptors and synchronisation of contractions (Garfield et al., 1990), and acts in concert with IL-8 to remodel the cervix (reviewed in Kelly, 2002).

The onset of labour is associated with activation of the Nuclear Factor Kappa B (NFκB) transcription factor system in the amnion which plays a role in the expression of pro-inflammatory genes such as interleukin-8 (IL-8), interleukin-6 (IL-6) and cyclo-oxygenases 1 and 2 (COX-1 and COX-2). COX genes are also referred to as prostaglandin H synthase or PG synthase. The resulting inflammatory infiltrate (mediated by the cytokines) and increase in prostaglandin synthesis (mediated by the cyclo-oxygenases) leads to cervical ripening, fetal membrane rupture and myometrial contractions.

Five members of the NF-κB/Rel family have been identified in mammals: NF-κB1 (p50 and its precursor p105), NF-κB2 (p52 and its precursor p100), p65 (RelA), c-rel, and RelB. These proteins share a structurally conserved amino-terminal region termed the Rel homology domain (RHD). The RHD is responsible for dimerisation, DNA binding, and interaction with the inhibitors of kappa B (IκB) proteins. It also contains a nuclear localisation signal (NLS). In its active DNA-binding form NF-κB consists of heterogeneous dimers of various combinations of NF-κB subunits: each member of the NF-κB family, except for RelB, can form homodimers, as well as heterodimers with one another. The p65, c-rel and RelB proteins contain a carboxy-terminal non-homologous transactivation domain, which activates transcription from κB sites in target genes; in contrast, p50 and p52 proteins lack a transactivation domain. The various NF-κB dimers exhibit different binding affinities for specific κB sites (Kunsch et al., 1992, Phelps et al., 2000), and differentially stimulate transcription through distinct κB elements (Lin et al., 1995).

In resting cells, NF-κB dimers are normally sequestered in an inactive form in the cytoplasm by association with the inhibitory IκB proteins, which include IκBα, IκBβ and IκBε. The IκBs are characterised by the presence of multiple ankyrin repeats which mediate binding to the RHD and mask the NLS of NF-κB.

The major NF-κB signalling pathway, which is activated by pro-inflammatory stimuli and LPS, targets IκBα- and IκBβ-bound NF-κB (for review see Li and Verma 2002). p50/p65 dimers are the most abundant form of NF-κB in most cell types, and activation of IκBα-bound p50/p65 dimers is the best characterised pathway. In this ‘classical’ pathway, diverse stimuli trigger signal transduction cascades that ultimately converge on the activation of a specific IκB kinase (IKK). The IKK complex consists of several proteins, the main ones being IKKα (IKK1), IKKβ (IKK2), and NF-κB essential modulator (NEMO or IKKγ). The activated IKK complex phosphorylates IκBα at serines 32 and 36, which results in the poly-ubiquitination of IκBα at lysines 21 and 22. This modification targets IκBα for rapid degradation by the 26S proteasome. The degradation of the IκB inhibitor exposes the NLS of NF-κB resulting in translocation of the p50/p65 dimer to the nucleus where it can bind to κB sites in the promoter of target genes and promote transcription.

Most stimuli cause only the transient activation of NF-κB. The critical inhibitory step in NF-κB inactivation involves binding of newly synthesised IκBα to NF-κB in the nucleus. IκBα is quickly resynthesised following its degradation. The newly synthesised IκBα is localised in the nucleus and displaces NFκB from its DNA binding sites. IκBα contains leucine-rich nuclear export sequences (NES) (Johnson et al 1999), which then enable it to transport NF-κB back to the cytoplasm, thereby completing an autoregulatory post-induction repression.

In many cells nearly half of the NF-κB is sequestered by the other major IκB isoform, IκB (Whiteside et al., 1997). In contrast to IκBα, IκBβ is not NF-κB inducible and does not exert a rapid post-induction repression of NF-κB activity. Rather, IκBβ has been implicated in persistent NF-κB activation. Prolonged exposure to certain stimuli, such as LPS, leads to the long-term induction of NF-κB activity despite high levels of newly synthesised IκBα. Following stimulus-induced degradation, the newly synthesised IκBβ is un-phosphorylated and, in contrast to IκBα or the constitutively phosphorylated IκBβ, can interact with NF-κB bound to target promoters without displacing it from the DNA (Suyang et al., 1996). This interaction of un-phosphorylated IκBP with DNA-bound NF-κB is thought to protect NF-κB from nuclear export, and thus inhibition, by IκBα, and the outcome is a sustained NF-κB response.

PGs are a family of biologically active molecules having a diverse range of actions depending on the prostaglandin type and cell target. There is considerable evidence to support a central role for PGs in human parturition. Labour is associated with increased PG synthesis within the uterus (Turnbull 1977) particularly from the fetal membranes (Skinner and Challis 1985). PGs act to mediate cervical ripening and to stimulate uterine contractions (Cranckshaw and Dyal 1994) and indirectly to increase fundamentally dominant myometrial contractility by up-regulation of oxytocin receptors and synchronisation of contractions (Garfield et al 1990). PG synthesis in amnion, chorion-decidua and myometrium increases with labour (for a review, see Bennett and Slater 1996). Chorion prostaglandin dehydrogenases are thought to protect the uterus from basal prostaglandin synthesis during pregnancy but are down-regulated at term. Deficiency of prostaglandin dehydrogenase in chorion has been associated with pre-term labour (van Meir 1996, 1997).

Accordingly, inhibition of prostaglandin synthesis is an effective method of preventing or arresting pre-term labour (Keirse, 1995). Conversely, prostaglandins have been administered to induce labour as a means to terminate pregnancy (Ganstrom et al, 1987).

Most PGs bind to prostanoid receptors localised on the cell surface and act through second messenger systems (Narumiya, 1995). However, PGD₂ metabolites are actively incorporated into the nuclei of cells (Narumiya et al., 1987) and can exert their effects through direct interactions with nuclear receptors. Peroxisome proliferator-activated receptors (PPARs) are ligand-activated transcription factors belonging to the nuclear receptor superfamily. They exist in three distinct forms, PPAR-α, PPAR-δ, and PPAR-γ, which form heterodimers with the retinoic X receptor (RXR) and bind to PPAR response elements (PPREs) in the promoter of target genes to induce transcription. PPAR-γ can also repress gene transcription by negatively interfering with the NF-κB, AP-1, STAT and C/EBP pathways (Zhou et al., 1999; Subbaramaiah et al., 2001; Takata et al., 2002; Suzawa et al., 2003).

The aetiology of pre-term labour is multi-factorial but bacterial infection is believed to play an important role, especially at earlier gestational ages (for review see Romero et al., 2002). A growing body of epidemiological data suggests that intrauterine infection is an important cause of brain injury in infants born before 32 weeks of gestation. During ascending intrauterine infection, micro-organisms can stimulate the production of pro-inflammatory cytokines, such as tumour necrosis factor α (TNFα) and IL-1β, as well as PGs and other inflammatory mediators, resulting in the premature onset of labour. Intrauterine infection/inflammation has also been identified as a key contributor to the development of cerebral palsy (CP) and schizophrenia (Urakubo et al., 2001; Gibson et al., 2003), and, although CP does occur in term infants, the risk of CP is strongly associated with prematurity (Dammann et al., 1999).

In addition, inflammatory responses caused by mechanical stretching of the uterus may contribute to the onset of labour. Mechanical stretching of the uterus occurs to an extent as a normal part of pregnancy and may be responsible for some of the biochemical changes which occur near to term and which cause the normal onset of labour at term. In the context of preterm labour, mechanical stretch may occur where the uterus is overdistended by multiple pregnancy or by excess amniotic fluid (clinically termed hydramnios or polyhydramnios). There may also be more local stretch of the lower segment of the uterus, the cervix and overlying fetal membranes in cases where there is cervical weakness (clinically termed cervical incompetence). Stretch leads to an increase in the production of a series of ‘labour-associated’ proteins including COX-2 (which then increases prostaglandin synthesis), cytokines such as IL-8 and IL-1b and the oxytocin receptor. Increased prostaglandin and cytokine productions causes cervical ripening or further cervical ripening (and may lead to neonatal brain injury). Prostaglandins and OTR receptor lead to uterine contractions.

Obstetric management of pre-term labour is still largely reactive and centred on the use of drugs intended to inhibit contractions to delay delivery. This was thought to be principally dependent upon gestational age leading to the concept that prolongation of the pregnancy will always improve outcome. However, there is now growing evidence that the mechanisms leading to pre-term birth also cause fetal cerebral damage. Characteristically, damage is localised to the white matter, involving both a diffuse astrogliosis with subsequent loss of myelin-producing oligodendrocytes, as well as multifocal necroses resulting in cystic change (periventricular leucomalacia, PVL). Such lesions lead to cerebral palsy in 60-90% of affected infants (described in Vlope, 2001).

There are currently no drugs available which will safely and effectively inhibit pre-term contractions. The most commonly used agents, β-sympathomimetics such as Ritodrine, Salbutamol and Terbutaline, cause significant maternal cardiovascular, respiratory and metabolic side effects and may lead to pulmonary oedema, cardiac failure and maternal death. Furthermore they are subject to tachyphylaxis and become ineffective after 24 to 48 hours. Meta-analysis of randomised controlled trials has shown that the value of β-sympathomimetics is only in the temporary delay of labour to allow in utero transfer or administration of steroid to improve fetal lung surfactant production.

Other than the antenatal administration of corticosteroids, no obstetric interventions affect neonatal outcome although improvements in neonatal intensive care have dramatically increased survival rates. Commonly used agents are dexamethasone or betamethasone. Antenatal administration of corticosteroids improves the outcome for the pre-term neonate since it reduces the incidence and severity of respiratory distress syndrome, intracranial haemorrhage and necrotising enterocolitis. One function of corticosteroids is to mature the fetal lung, which leads to an increase in surfactant production and therefore prevents or reduces the severity of neonatal respiratory problems. Such agents are known to those skilled in the art.

Current obstetric management of pre-term labour (or threatened pre-term labour or pre-term premature rupture of membranes) is to attempt to delay delivery using ‘tocolytic’ drugs to allow time for steroid administration.

Typically, effective tocolytic drugs are oxytocin receptor antagonists, calcium channel blockers, sympathomimetics and nitric oxide donors.

A commonly used oxytocin receptor antagonist is Atosiban, that functions by blocking the oxytocin receptor, thereby preventing activation of the receptor by endogenous oxytocin that stimulates uterine contractions. A commonly used calcium channel blocker is Nifedipine, that functions to block the influx of calcium into the myometrial cells, which is a requirement for contractions to take place. A commonly used sympathomimetic is Ritodrine, that functions by activating adrenergic receptors on the myocyte cell membrane leading to phosphorylation and down-regulation of the activity of myosin light chain kinase, an enzyme essential for contractions. A commonly used nitric oxide donor is glyceryl trinitrate, that functions by increasing myocyte cGMP thereby down-regulating the activity of myosin light chain kinase, an enzyme essential for contractions.

Indomethacin, a cyclo-oxygenase inhibitor, is effective in preventing the contractions of pre-term labour. It is more effective in short term prolongation of pregnancy than the β-sympathomimetics and, unlike β-sympathomimetics, it can reduce the risk of delivery pre-term (Keirse 1995). The use of indomethacin is limited by fetal side effects. Indomethacin reduces fetal urine output and constriction of the ductus arteriosus (Moise et al 1995). Clinically significant ductal constriction occurs only in a proportion, increasing with gestational age from 10% at 26 weeks to 50% at 32 weeks. Accordingly the use of indomethacin is limited in clinical practice to use ≦32 weeks, and to short courses (≦48 hours) after which any effects on the constriction of the ductus have been shown to be reversible (Tulzer et al 1991; Moise et al 1993; Respondek et al 1995).

Because of these side effects some obstetricians now use Sulindac, which appears to be equally good as a tocolytic (Carlon et al 1992) in place of indomethacin. Sulindac produces a smaller reduction in fetal urine output and minimal effect on ductal patency (Carlon et al 1992; Rasanen and Jouppila 1995). However, Sulindac is far from an ideal choice of tocolytic agent.

Accordingly, new agents or regimens capable of reducing and/or preventing an inflammatory response in the reproductive system of a female are highly desired. Such medicaments or approaches would allow the treatment of pathogenic infection within the reproductive system of a female and/or delay pre-term delivery without causing injury to the fetus/neonate.

In light of the above, the present inventors have surprisingly discovered that prostaglandins can be used to delay the onset and/or prevent the continuation of labour in a female.

Thus, in a first aspect, the present invention provides the use of a cyclopentenone prostaglandin in the manufacture of a medicament for delaying the onset and/or preventing the continuation of labour in a female.

Preferably, this is achieved by preventing and/or reducing an inflammatory response in the reproductive system of a female.

The invention stems from the unexpected finding that the cyclopentenone prostaglandins, such as 15-deoxy-Δ^{12,14}prostaglandin J₂ (15-dPGJ₂) and prostaglandin A₁ (PGA₁), inhibit and/or reduce NFκB activity within uterine cells of the female reproductive system. Thus, cyclopentenone prostaglandins provide a means for the inhibition and/or reduction of NFκB activity in the reproductive system of a female. Medicaments of the invention are believed to inhibit cytokine synthesis and inhibit the biochemical processes of labour, thereby safely prolonging pregnancy. Accordingly, the present invention will improve obstetric management of pre-term labour as the onset of labour may be delayed without injuring the fetus/neonate.

The cyclopentenone prostaglandins are naturally-occurring substances that contain a cyclopentenone ring structure. The cyclopentenone ring is characterised by the presence of a chemically-reactive α,β-unsaturated carbonyl and is formed by dehydration of the cyclopentane ring of a precursor prostaglandin.

Generally, the first step in the biosynthesis of prostaglandins involves the intracellular release of arachidonic acid from plasma membrane phospholipids via the action of phospholipase A₂. Arachidonic acid is then converted sequentially to PGG₂ and PGH₂ by the cyclo-oxygenase and peroxidase activities of the PGH synthases, PGH 1 and 2. The prostaglandins PGE₂, PGD₂ and PGF_{2α} are subsequently synthesised from PGH2 via the action of the PGE₂, PGD₂ and PGF_{2α} synthase, respectively. The cyclopentenone prostaglandins, prostaglandin A₂ (PGA₂), prostaglandin A₁ (PGA₁) and prostaglandin J₂ (PGJ₂) are formed by dehydration of prostaglandin E₂ (PGE₂), prostaglandin E₁ (PGE₁) and prostaglandin D₂ (PGD₂), respectively. PGJ₂ is metabolised further to Δ¹²-prostaglanding J₂ (Δ¹²-PGJ₂), and 15-deoxy-Δ^{12,14}prostaglandin J₂ (15-dPGJ₂).

Other unnatural or synthetic prostaglandins can be made by chemical synthesis. Total synthesis of prostaglandins was first accomplished by Corey in the 1960s (reviewed in Corey, 1991), and subsequently simplified by Suzuki et al. (1990). This latter scheme uses a C8 organometallic reagent for one side chain and a C7 acetylenic halide for the other side chain which are added to the desired chemical head-group. This synthesis is versatile and allows the synthesis of a variety of natural and unnatural prostaglandins including the cyclopentenone prostaglandins. A general pathway for natural and chemical synthesis of prostaglandins and cyclopentenone prostaglandins is described in Straus and Glass (2001), the disclosure of which is incorporated herein.

Chemical modification of cyclopentenone prostaglandins using techniques known in the art of chemistry may alter the clinical effectiveness of the molecule. Such alterations may, for example, increase or decrease the stability or another characteristic of the cyclopentenone prostaglandin, to give a desired change in activity. For example, modification of the 15 C residue of cyclopentenone prostaglandins will reduce the metabolism of the compound, thereby increasing its half-life in vivo. Such modifications will be appreciated by those skilled in the art.

Thus, by “cyclopentenone prostaglandin”, we include any natural, unnatural or chemically-modified prostaglandin which has a cyclopentenone ring. Cyclopentenone prostaglandin is often abbreviated to “cyPG”. Especially preferred cyclopentenone prostaglandins include prostaglandin D₂ (PGD₂) and its metabolite 15-deoxy-Δ^{12,14}prostaglandin J₂ (15-dPGJ₂). Also preferred is prostaglandin A₁ (PGA₁).

15-dPGJ₂ may be obtained from Cayman Chemical, 1180 East Ellsworth Road, Ann Harbour, Mich. 48108 USA (catalogue number 18570). PGA, may be obtained from Alexis Biochemicals Ltd, PO Box 6757, Bingham, Nottingham, NG13 8LS, UK (catalogue number 340-045-M005).

By “onset of labour” and/or “continuation of labour” we include the biochemical and/or physiological changes associated with preparation of the tissues of the female reproductive system for delivery. For example, the uterus increases in contractility and undergoes contractions. The cervix also ripens in readiness for delivery. Such changes are well known in the arts of obstetrics, gynecology and midwifery and, for example, the Bishop's score indicates the degree of cervical ripening (described in Herman et al., 1993). By “delaying the onset of labour in a female and/or preventing the continuation of labour in a female” we include the meaning that at least one of these biochemical and/or physiological changes are delayed or prevented.

By “female” we include any female mammal such as human, or a domesticated mammal, preferably of agricultural significance including a horse, pig, cow, sheep, dog and cat. It is preferred if the female is a human female.

In a second aspect, the present invention provides the use of a cyclopentenone prostaglandin in the manufacture of a medicament for preventing and/or reducing an inflammatory response in the reproductive system of a female. Such medicaments are able to inhibit and/or reduce NFκB activity in uterine cells.

By “NFκB” we include homo- and heterodimers of RelA (p65), RelB, NFκB1 (p50), NFκB2 (p52) and cRel. The RelA (p65), RelB, NFκB1 (p50), NFκB2 (p52), and cRel genes and the sequence of the polypeptide products are described in Li et al. (2002).

By “NFκB activity” we include the activities of NFκB associated with the expression of genes controlled by any homo- or heterodimer of RelA (p65), RelB, NFκB1 (p50), NFκB2 (p52) or cRel of the NFκB transcription factor family. In particular, we include: nuclear translocation of NFκB which can be measured, for example, by Western blotting analysis of nuclear and cytosolic cellular fractions for the protein of interest (described in Sambrook et al., 1989; Lee et al., 2003); binding of NFκB to target nucleic acid sequences (such as specific regions and sequences of DNA), which can be measured, for example, by Electro-Mobility Shift Assay (EMSA, as described in Dignam et al., 1983; Lee et al., 2003); and NFκB-mediated expression of target genes which can be measured, for example, by northern blotting and/or Western blotting (Sambrook et al., 1989; Lee et al., 2003). Methods for measuring these activities of NFκB are well known by those skilled in the art of biochemistry and molecular biology.

By “uterine cells” we include any cells within the uterus of a female, or cells derived from the uterus of a female, particularly placental cells, amnion cells, myocytes, uterine and cervical fibroblasts, and maintained as a primary or transformed cell culture or line. These cell types are typically referred to as “gestational tissues”.

Cultures of amnion cells may be prepared from tissue by separating the entire amnion, except for the part overlying the placenta, from the chorion, followed by separating amnion epithelial cells from fibroblasts and maintaining the epithelial cells using mammalian cell culture techniques (Lee et al., 2003). Myometrial cell culture may be prepared from tissue from the lower uterine segment, separating cells by incubation with Dispase and collagenase/elastase/DNAase solution and maintaining the myometrial cells using mammalian cell culture techniques (Pieber et al., 2001). Techniques for the generation and maintenance of primary and transformed mammalian cell cultures will be well known to those skilled in the relevant art.

By “reproductive system of a female”, we include any cells and/or tissues and/or organs of a female directly or indirectly involved in the formation, nourishment, maintenance and development of a neonate, embryo or fetus at any gestational stage during pregnancy. In particular we include the cells and/or tissues of the uterus, placenta, amnion, chorion, decidua, cervix and vagina.

Preferably, the medicament is for preventing and/or reducing an inflammatory response in the reproductive system of a female that is pregnant.

By “inflammatory response” we include biochemical and physiological changes associated with inflammation mediated by cells of the host's immune system. Such changes are known in the arts of human and veterinary medicine, immunology, molecular biology and biological science.

If a patient is detected clinically at being at high risk of preterm delivery, because of detection of fibronectin in the vagina, identification of cervical shortening on ultrasound, the identification on clinical examination of cervical dilatation, or the onset of contractions then there is a high risk that there may be inflammation within the uterus. Other clinical measures of inflammation within the uterus are maternal temperature, white blood cell count, serum c-reactive protein concentrations and amniotic cytokine concentrations (taken at amniocentesis) which suggest a high risk of inflammation within the uterus if abnormal. Methods for measuring such changes will be well known to those skilled in the art.

By “pregnant”, we include the meaning that the female is carrying a fertilised egg in the uterus, or an embryo or neonate or fetus at any stage of gestational development.

Preferably, the present invention provides a use wherein the female is human and the duration of pregnancy is more than approximately 13 weeks of human pregnancy. More preferably, the duration of pregnancy is approximately between 20 and 32 weeks.

Preferably, the medicament reduces and/or prevents an inflammatory response in the reproductive system of a female associated with the onset or continuation of labour. The biochemical and physiological changes associated with the onset or continuation of labour have been mentioned above.

There are many situations where it is useful to substantially prevent or reduce at least one of the changes in the female reproductive system associated with the onset or continuation of labour. For example, it is well known that certain groups of pregnant females are at high risk of pre-term labour. Females that have had one or more instances of pre-term labour previously are at considerably higher risk of a further pre-term labour when pregnant. An increased risk of pre-term labour can also be determined by measuring oncofetal fibronectin levels and by cervical examination using methods well known in the art.

It is also useful to prevent or reduce at least one of the changes in the female reproductive system associated with the continuation of labour, particularly uterine contractions, temporarily in circumstances where this is desirable. For example, it may be desirable temporarily to inhibit uterine contractions during labour in order to clear the fetal lungs or in order to transfer the female from one place to another. It is often desirable to transfer the female to a more suitable place where better care is available for her and the offspring.

It is also useful to substantially prevent for a considerable duration pre-term labour using the method of the invention. In particular, it is useful to inhibit pre-term uterine contractions from the time when they first occur (or soon thereafter) until the normal time of delivery.

Preferably, the medicament reduces and/or prevents an inflammatory response in the reproductive system of a female associated with injection by a pathogenic agent

More preferably, the pathogenic agent is viral, bacterial or fungal.

Preferably, the medicament reduces and/or prevents an inflammatory response in the reproductive system of a female associated with stretch of the uterus.

By “stretch of the uterus” we include mechanical stretching of the uterus occurring where the uterus is overdistended by multiple pregnancy or by excess amniotic fluid (clinically termed hydramnios or polyhydramnios). There may also be more local stretch of the lower segment of the uterus, the cervix and overlying fetal membranes in cases where there is cervical weakness (clinically termed cervical incompetence).

Preferably, the medicament reduces and/or prevents one or more of the following conditions: pre-term labour; pathogenic infection; cervical ripening, uterine contractions.

By “pre-term labour”, we include the meaning of spontaneous labour occurring before the usual calculated time for delivery. In humans, pre-term labour is defined as spontaneous labour occurring before 37 weeks of gestation (with 39 weeks being term). The usual calculated time of delivery for females as defined by the invention will be well known in the arts of human and veterinary medicine.

Preferably, the medicament reduces and/or prevents fetal or neonatal damage.

More preferably, the medicament reduces and/or prevents one or more of the following conditions: astrogliosis; loss of myelin-producing oligodendrocytes; multifocal necroses resulting in cystic change (periventricular leucomalacia, PVL).

By “astrogliosis” we include the meaning of hypertrophy (i.e. increasing cell size) of the astroglia, that usually occurs in response to injury. Astroglia are the largest and most numerous neuroglial cells in the brain and spinal cord. Astrocytes (from “star” cells) are irregularly shaped with many long processes, including those with “end feet” which form the glial (limiting) membrane and directly and indirectly contribute to the blood-brain barrier. They regulate the extracellular ionic and chemical environment, and “reactive astrocytes” (along with microglia) respond to injury. Astrocytes can release neuro-transmitters, but their role in signaling (as in many other functions) is not well understood.

By “oligodendrocytes” we include the meaning of neuroglial cell of the central nervous system (CNS) in vertebrates whose function is to myelinate CNS axons. “Loss of myelin-producing oligodendrocytes” means that there a reduction in the number of these cells.

By “multifocal necroses” we include the meaning of death of tissue occurring at more than one site. By “cystic change” we include the meaning of the development of fluid filled spaces in the region where necrosis has taken place. By “periventricular leucomalacia” or “PVL” we include the meaning of damage to the periventrical cerebral white matter which is seen following cytokine induced or hypoxia/ischemia induced necroses and which can go on to become cystic change.

A particularly preferred embodiment of the invention is the use of the cyclopentenone prostaglandin 15-deoxy-Δ^{12,14}-prostaglandin J₂ and/or prostaglandin A₁.

Alternatively, the cyclopentenone prostaglandin is provided in the form of a prodrug of 15-deoxy-Δ^{12,14}-prostaglandin J₂ and/or prostaglandin A₁.

It will be appreciated by those skilled in the art that certain metabolic precursors of cyclopentenone prostaglandins, may not possess pharmacological activity as such, but may, in certain instances, be administered to a patient and thereafter metabolised in the body to form compounds of the invention which are pharmacologically active. Such derivatives may therefore be described as “prodrugs”.

All prodrugs of the cyclopentenone prostaglandins, particularly those of 15-deoxy-Δ^{12,14}-prostaglandin J₂ and/or prostaglandin A₁, are included within the scope of the invention.

Preferably, the prodrug is PGD₂ (the precursor of 15-dPGJ₂) or PGE₁ (the precursor of PGA₁).

Preferably, the medicament further comprises a pharmaceutically acceptable excipient, diluent or carrier.

By “pharmaceutically acceptable” we mean that the carrier does not have a deleterious effect on the recipient. Typically, the carrier will be sterile and pyrogen free.

Preferably the medicament is in a form adapted for delivery by mouth, intravenous injection or intra-amniotic injection.

Preferably, the medicament is in a form which is compatible with the amniotic fluid. More preferably, the medicament is in a form which has substantially the same pH and/or osmotic tension as amniotic fluid.

The amniotic fluid has a distinct pH and a distinct osmotic tension. The amniotic fluid pH and osmotic tension are well known to, or can be readily measured by, the person skilled in the art.

Preferably, the medicament further comprises an agent for treating a female who has or is at risk of one or more of the following conditions: pre-term labour; pathogenic infection; cervical ripening, uterine contractions.

By an “agent for treating a female who has or is at risk of one or more of the following conditions: pre-term labour; pathogenic infection; cervical ripening, uterine contractions” we include corticosteroids, tocolytic agents and anti-inflammatory prostaglandins.

Preferably, the agent is a corticosteroid.

More preferably, the agent is capable of preventing and/or reducing respiratory distress syndrome.

One function of corticosteroids is to mature the fetal lung, which leads to an increase in surfactant production and therefore prevents or reduces the severity of neonatal respiratory problems

More preferably, the agent is selected from dexamethasone or betamethasone. Such agents are known to those skilled in the art. Administration of such agents may be two doses of 12 mg intramuscular (IM), 12 or 24 hours apart.

Preferably, the agent is capable of delaying delivery.

More preferably, the agent capable of delaying delivery is selected from: oxytocin receptor antagonists; calcium channel blockers; sympathomimetics; nitric oxide donors

Preferably, the agent is a tocolytic agent.

By “tocolytic” we include the meaning of a drug whose action is to stop uterine contractions.

More preferably, the tocolytic agent is selected from: oxytocin receptor antagonists, calcium channel blockers, sympathomimetics, nitric oxide donors.

More preferably, the oxytocin receptor antagonist is Atosiban. More preferably, the calcium channel blocker is Nifedipine. More preferably, the sympathomimetic is Ritodrine. More preferably, the nitric oxide donor is glyceryl trinitrate.

Preferably, the inflammatory response is mediated by NFκB in uterine cells.

More preferably, the cyclopentenone prostaglandin is capable of inhibiting and/or reducing NFκB activity by preventing and/or reducing NFκB DNA-binding in uterine cells.

More preferably, the cyclopentenone prostaglandin is capable of inhibiting and/or reducing NFκB activity by preventing and/or reducing NFκB-mediated transcriptional regulation in uterine cells.

More preferably, the cyclopentenone prostaglandin is capable of inhibiting and/or reducing NFκB activity by preventing and/or reducing NFκB production in uterine cells.

A further aspect of the invention is to provide a pharmaceutical composition comprising a cyclopentenone prostaglandin and a pharmaceutically acceptable carrier or excipient, the cyclopentenone prostaglandin being present in an amount effective to prevent and/or reduce an inflammatory response in the reproductive system of a female.

A further aspect of the invention is a method of treating inflammation within the reproductive system of a female, the method comprising administering an effective amount of a medicament of the invention.

Preferred, non-limiting examples which embody certain aspects of the invention will now be described, with reference to the following figures:

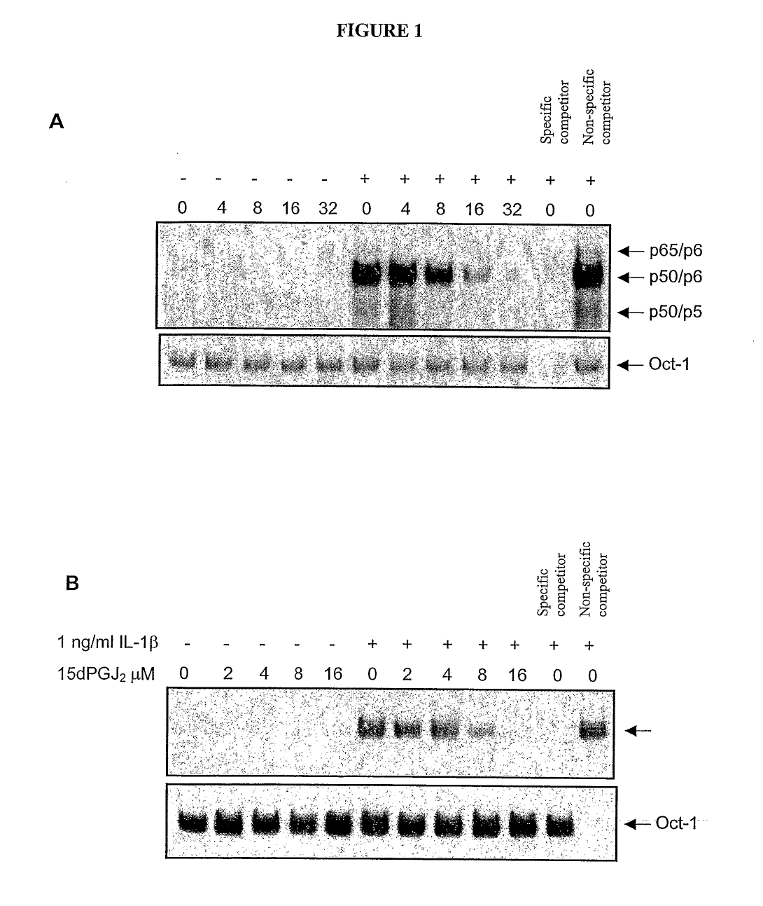

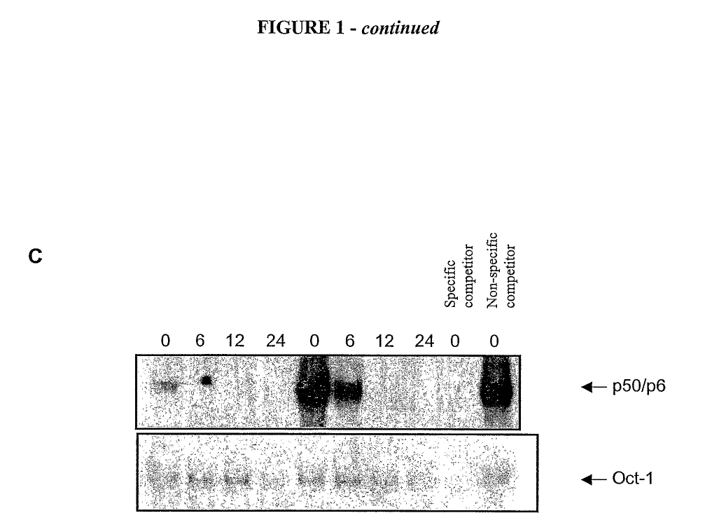

FIG. 1: 15dPGJ₂ inhibition of NF-κB DNA binding

Electro-mobility shift assay (EMSA) analysis of NF-κB DNA binding in nuclear protein extracts from (A) myometrial cells, (B) L+amnion cells, and (C) L− amnion cells treated with 15dPGJ₂ or vehicle for 2 h+/−IL-1b stimulation (15 min). Consensus kB probe used to assess NF-κB DNA binding, and consensus Oct-1 probe used as control.

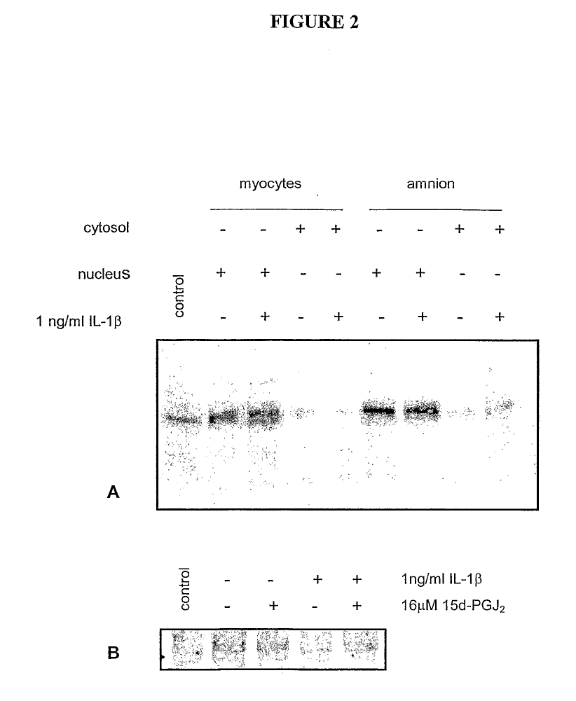

FIG. 2: PPAR-γ protein expression

Western immunoblots of (A) nuclear and cytosolic protein extracts from myometrial and amnion cells with or without 15 min IL-1β stimulation, and (B) nuclear extracts of myometrial cells treated with 15d-PGJ₂+/−IL-1b. Probing with antibody to PPARγ.

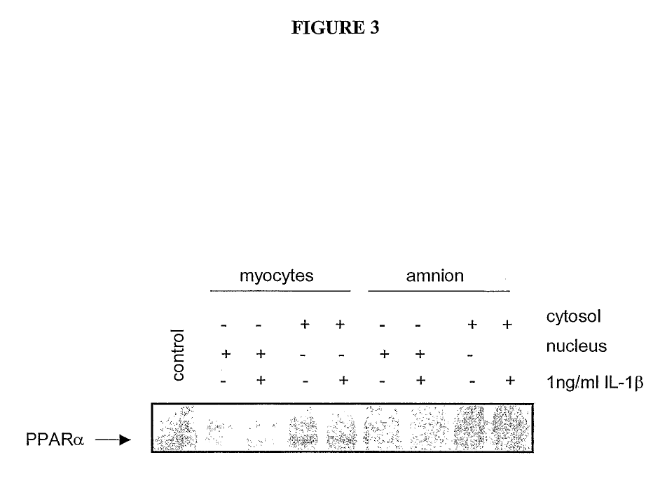

FIG. 3: PPAR-α protein expression

Western immunoblots of nuclear and cytosolic protein extracts from myometrial and amnion cells with or without 15 min IL-1b stimulation. Probing with antibody to PPARα.

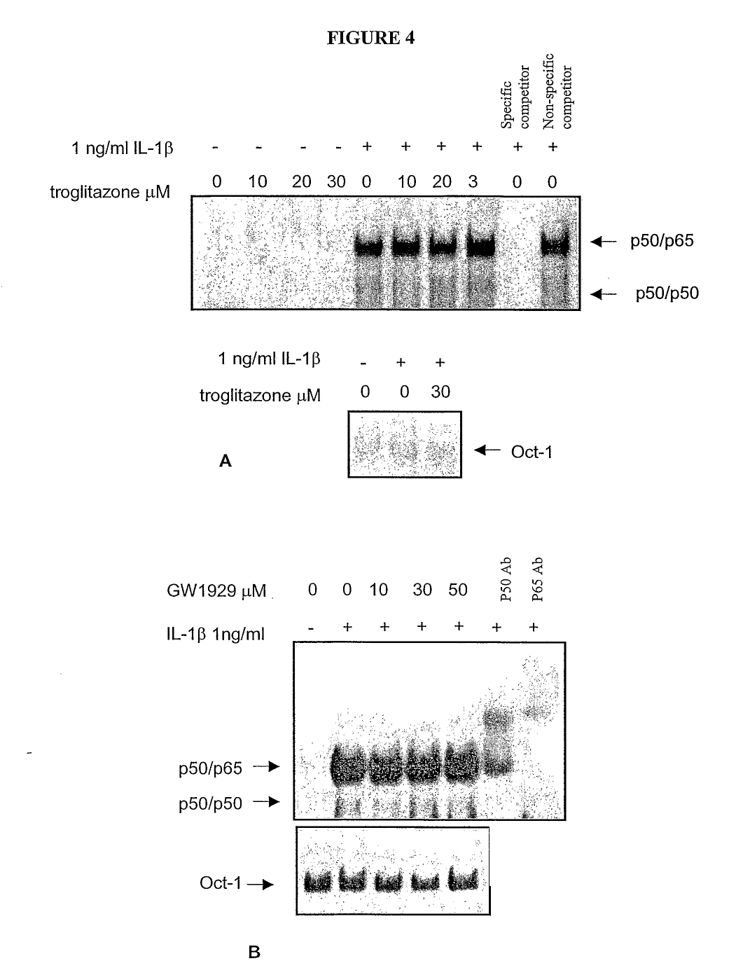

FIG. 4: PPAR-γ agonists do not inhibit NFkB DNA binding

EMSA analysis of nuclear protein extracts from myometrial cells treated with (A) troglitazone, (B) GW1929 or vehicle for 2 h+/−IL-1b stimulation (15 min). Consensus κB probe used to assess NF-κB DNA binding, consensus Oct-1 probe used as control. For supershift analysis, extracts were preincubated with antibodies against p50 or p65.

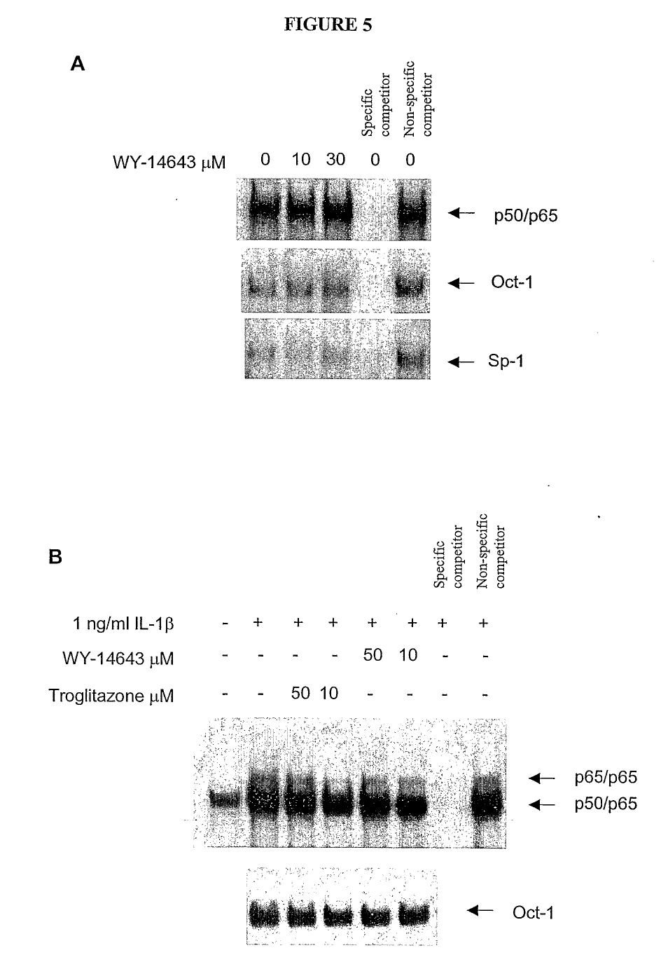

FIG. 5: Troglitazone and WY-14643 do not inhibit NFκB DNA binding

EMSA analysis of nuclear protein extracts from myometrial cells treated with (A) WY-14643 or vehicle, and (B) high doses of troglitazone, WY-14643 or vehicle for 2 h followed by IL-1b stimulation (15 min). Consensus κB probe used to assess NFκB DNA binding, consensus Oct-1 and Sp-1 probes used as controls.

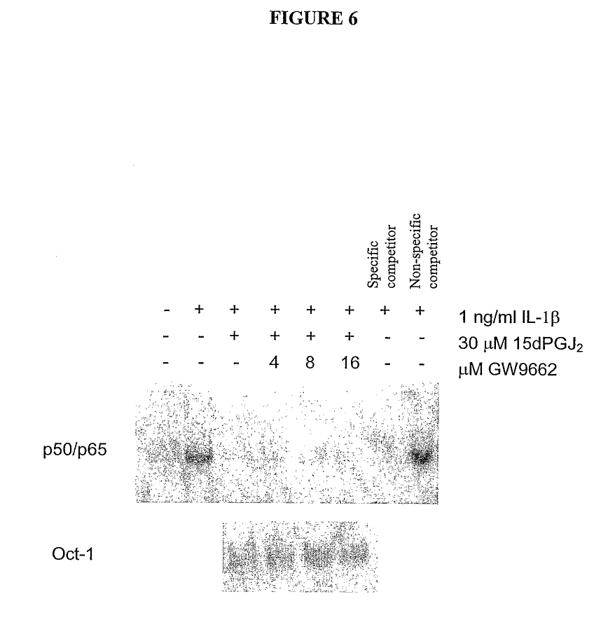

FIG. 6: PPAR-γ antagonist GW9662 does not alleviate 15dPGJ₂ inhibition of NFκB DNA binding

EMSA analysis of nuclear protein extracts from amnion cells treated with 15dPGJ₂+/−GW9662 or vehicle for 2 h followed by IL-1β stimulation (15 min). Consensus kB probe used to assess NFκB DNA binding, consensus Oct-1 probe used as control.

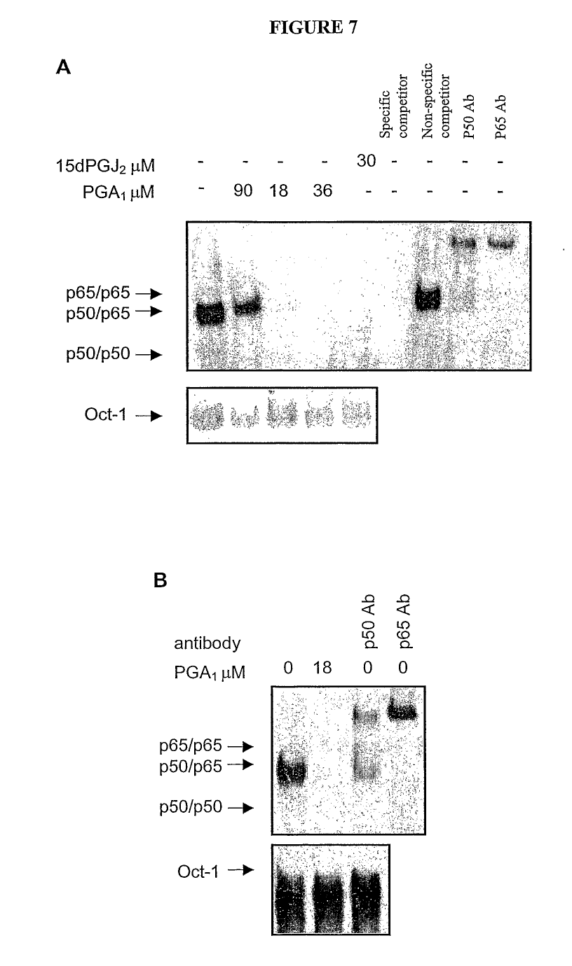

FIG. 7: PGA₁ inhibition of NFκB DNA binding

EMSA analysis of nuclear protein extracts from (A) myometrial cells, and (B) amnion cells treated with PGA₁ or vehicle for 2 h followed by IL-1b stimulation (15 min). Consensus κB probe used to assess NFκB DNA binding, consensus Oct-1 probe used as control. For supershift analysis, extracts were preincubated with antibodies against p50 or p65.

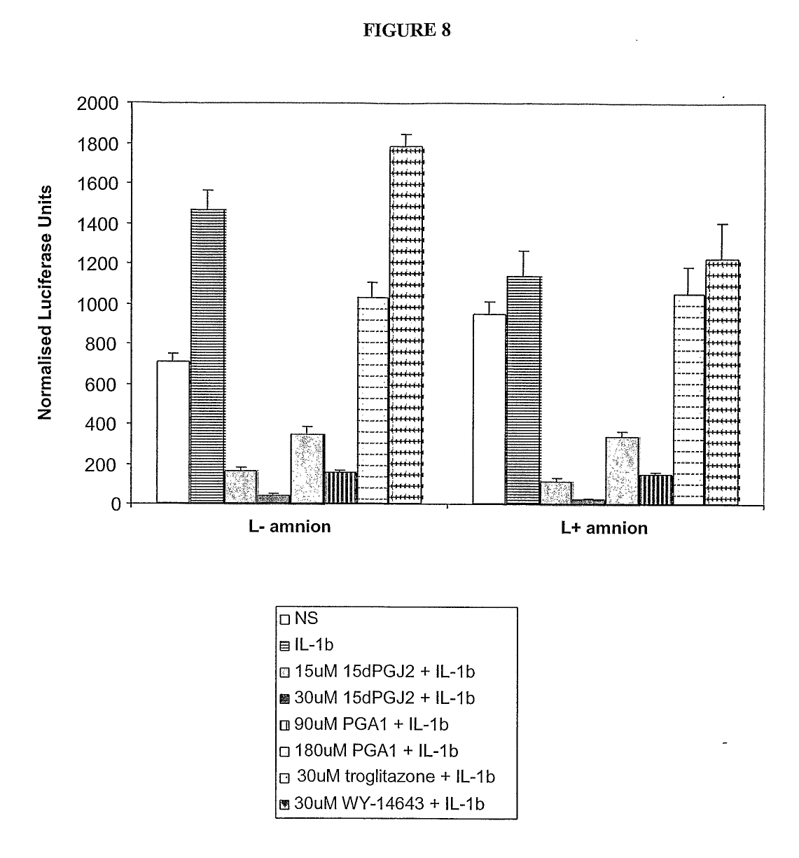

FIG. 8: Effect of cyPGs and PPAR agonists on NFκB transcriptional activity in amnion

Amnion cells derived from L− or L+ placentas were transiently transfected with the NFκB-dependent reporter construct κB.BG.Luc, treated with 15dPGJ₂, PGA₁, troglitazone, WY-14643, or vehicle for 2 h, and then stimulated with IL-1β (1 ng/ml) for 6 h. The mutated κBmut.Luc construct was used as a control to confirm NFκB-mediated transactivation. Values are normalised for b-gal reporter activity.

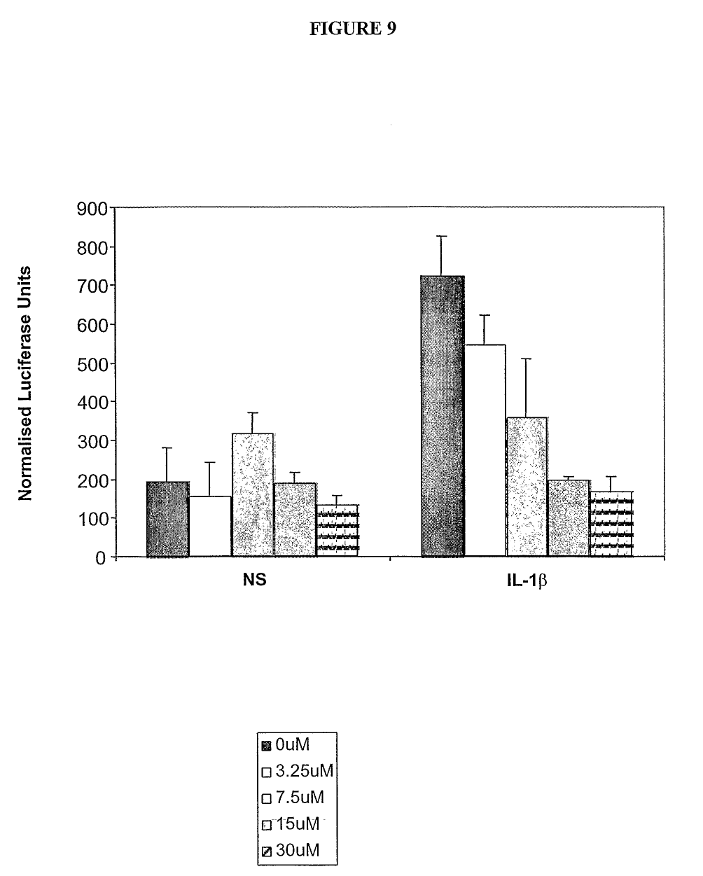

FIG. 9: 15dPGJ₂ inhibition of NFκB transcriptional activity in myometrium

Myometrial cells were transiently transfected with the NFκB-dependent reporter construct κB.BG.Luc, treated with 15dPGJ₂ or vehicle for 2 h, +/−IL-1β(1 ng/ml) for 6 h. The mutated κBmut.Luc construct was used as a control to confirm NFκB-mediated transactivation. Values are normalised for b-gal reporter activity. (NS=nonstimulated).

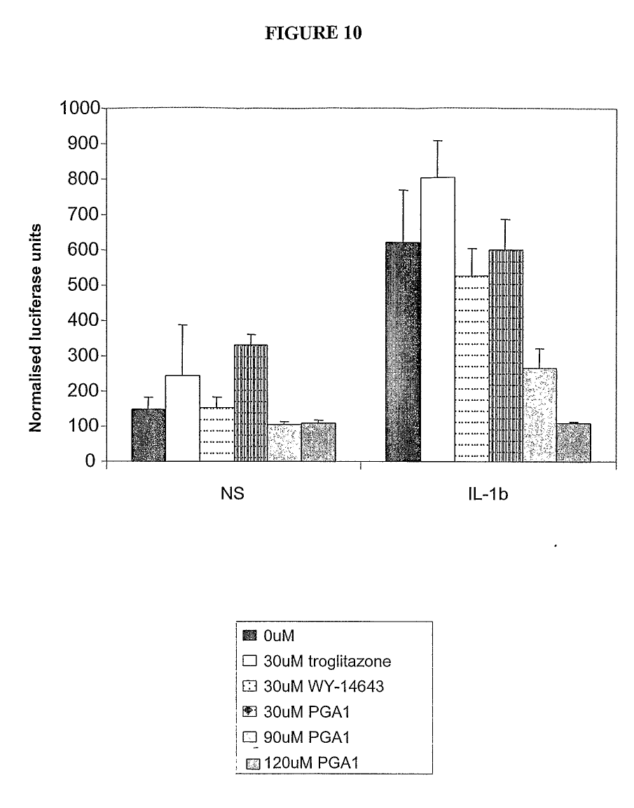

FIG. 10: Effect of PGA₁ and PPAR agonists on NFκB transcriptional activity in myometrium

Myometrial cells were transiently transfected with the NFκB-dependent reporter construct κB.BG.Luc, treated with troglitazone, WY-14643, PGA₁ or vehicle for 2 h, +/−IL-1b (1 ng/ml) for 6 h. The mutated κBmut.Luc construct was used as a control to confirm NFκB-mediated transactivation. Values are normalised for CMV-Renilla reporter activity. (NS=nonstimulated).

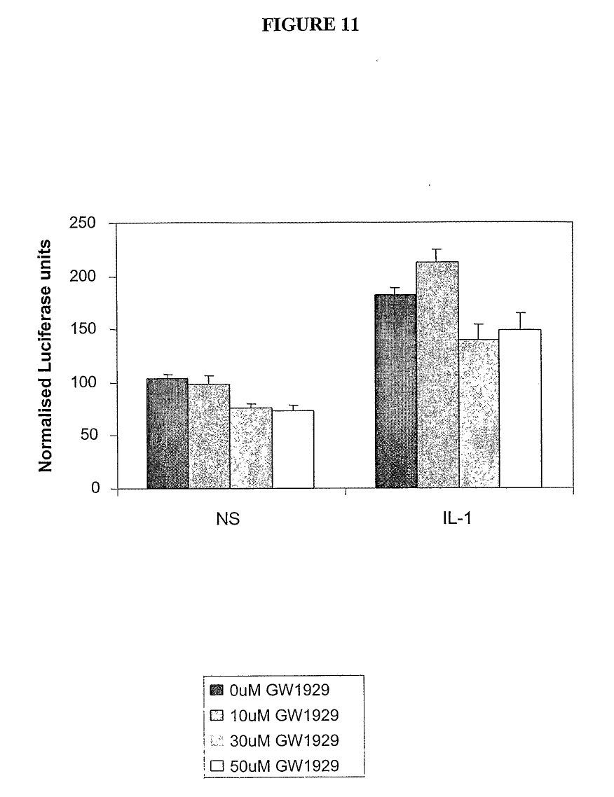

FIG. 11: PPAR-γ agonist GW1929 does not inhibit NFκB transcriptional activity

Myometrial cells were transiently transfected with the NFκB-dependent reporter construct κB.BG.Luc, treated with GW1929 or vehicle for 2 h, +/−IL-1b (1 ng/ml) for 6 h. Values are normalised for CMV-Renilla reporter activity. (NS=nonstimulated).

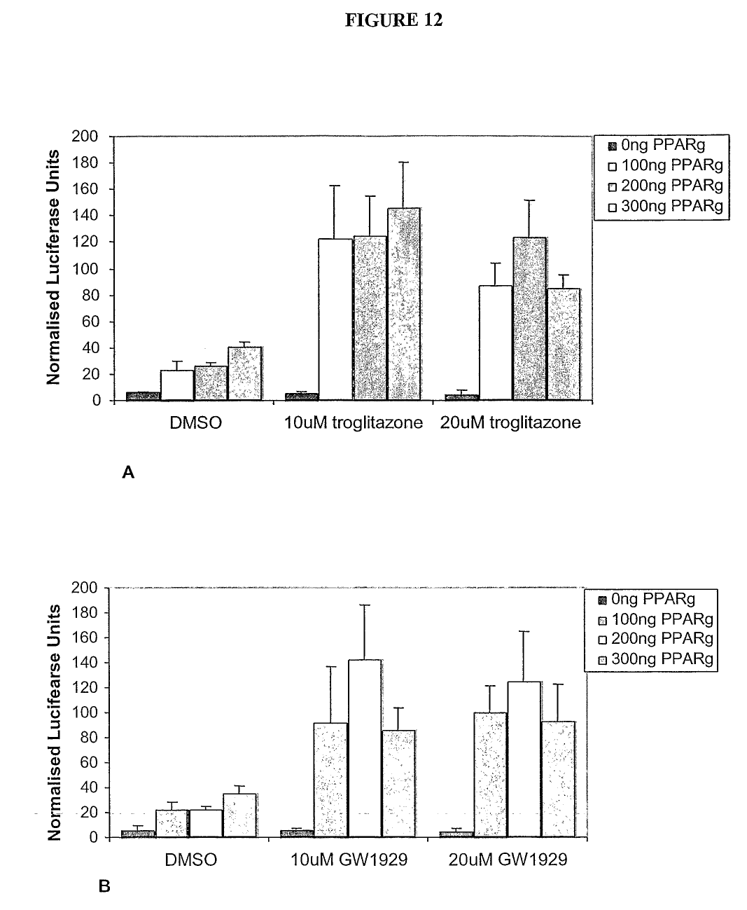

FIG. 12: Troglitazone and GW1929 potentiate PPAR-γ activation of a PPRE reporter

Myometrial cells were cotransfected with 0.4 mg of the PPAR-γ-dependent reporter construct 3-PPRE-TK.pGL3 and 100 ng, 200 ng or 300 ng of a PPAR-γ expression construct. Cells were treated with 10 mM or 20 mM of (A) troglitazone or (B) GW1929, or vehicle for 24 h. Values are normalised for CMV-renilla reporter activity. Similar results were obtained with transfection of amnion cells.

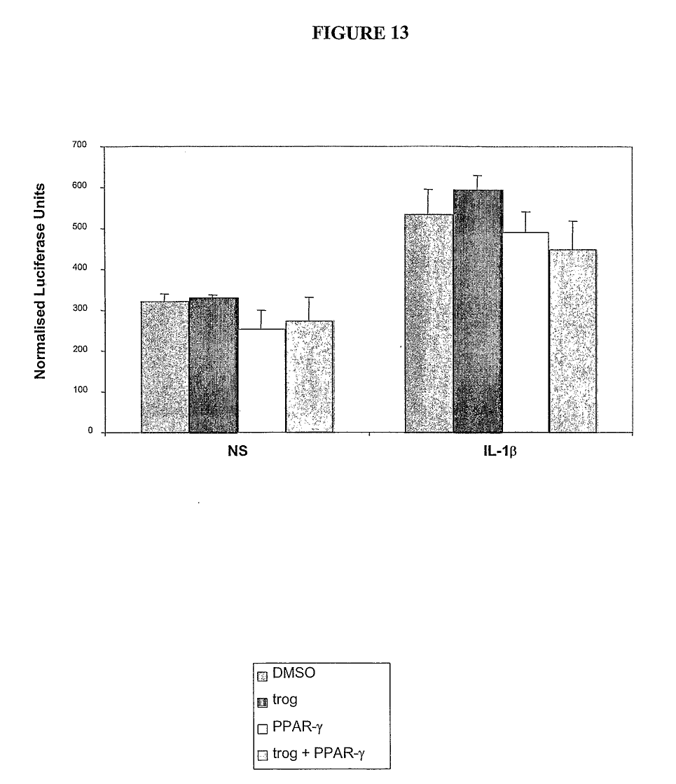

FIG. 13: Troglitazone does not inhibit NFκB transcriptional activity in PPAR-γ-transfected cells

Myometrial cells were transfected with 0.4 mg κB.BG.Luc reporter and 200 ng PPAR-γ expression vector and treated with 10 mM troglitazone or vehicle for 7 h+/−IL-1b (1 ng/ml) for 17 h. Values are normalised for CMV-renilla reporter activity. (NS=nonstimulated).

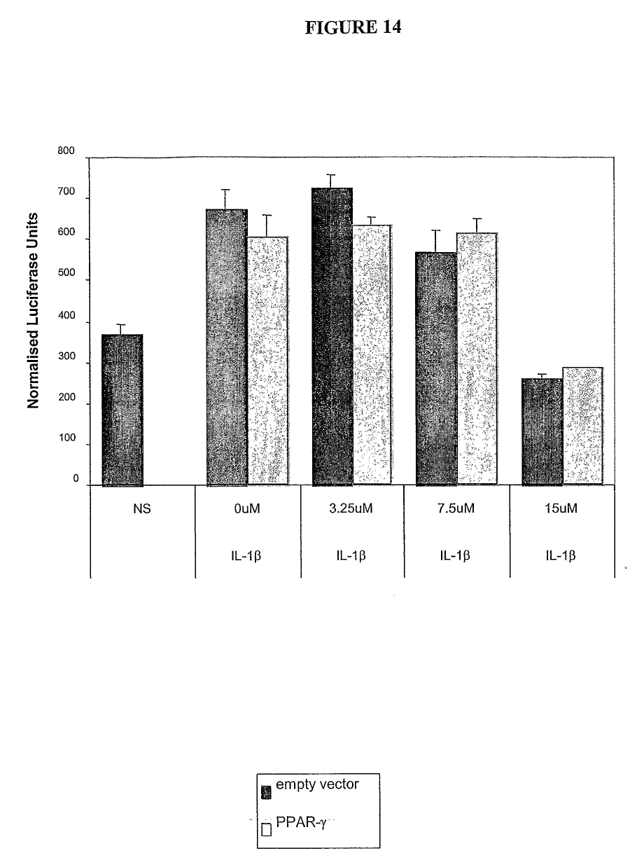

FIG. 14: PPAR-γ overexpression does not potentiate 15d-PGJ₂ inhibition of NFkB activity

Myometrial cells were transfected with 0.4 mg κB.BG.Luc reporter and 200 ng PPAR-γ expression vector and treated with 15d-PGJ₂ for 2 h followed by IL-1β (1 ng/ml) for 6 h. Values are normalised for β-galactosidase reporter activity. (NS=nonstimulated).

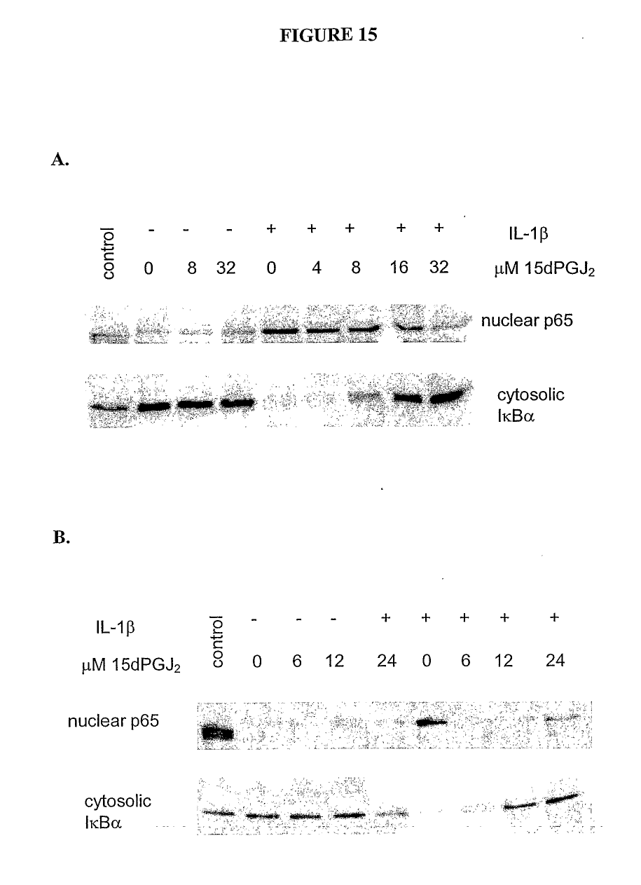

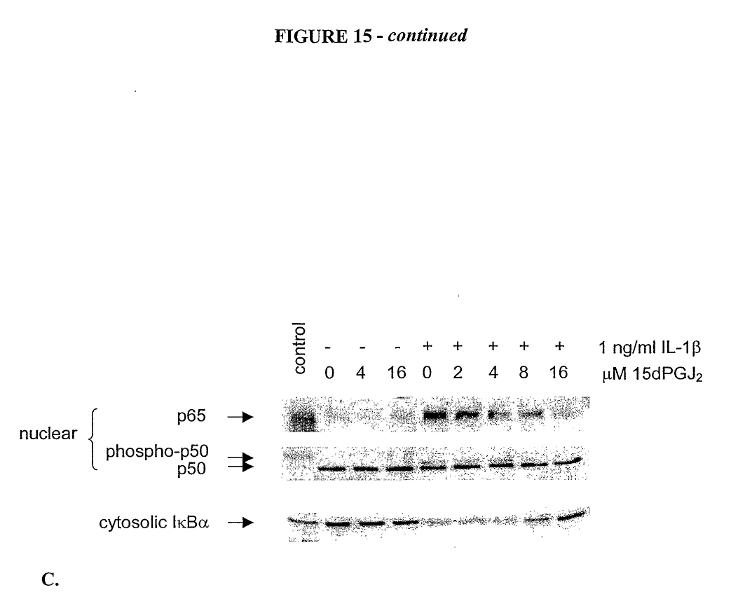

FIG. 15: 15dPGJ₂ inhibition of p65 nuclear localisation, p50 phosphorylation, and IκBα degradation

Western immunoblots of nuclear or cytosolic protein extracts from (A) myometrial cells, (B) L− amnion cells, and (C) L+amnion cells treated with 15dPGJ₂ or vehicle for 2 h+/−IL-1, stimulation (15 min). Blots probed with antibodies against p65, p50 or IκBα.

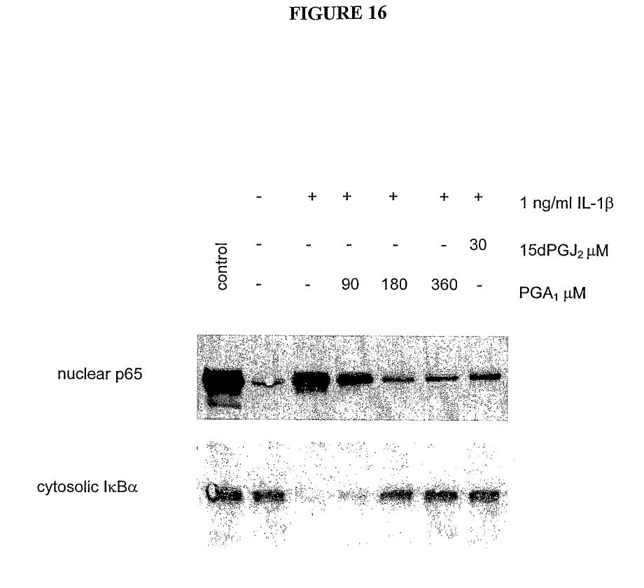

FIG. 16: PGA₁ inhibition of p65 nuclear localisation and IκBα degradation

Western immunoblot of nuclear or cytosolic protein extracts from myometrial cells treated with PGA₁ or vehicle for 2 h followed by IL-1β stimulation (15 min). Blots probed with antibodies against p65 or IκBa.

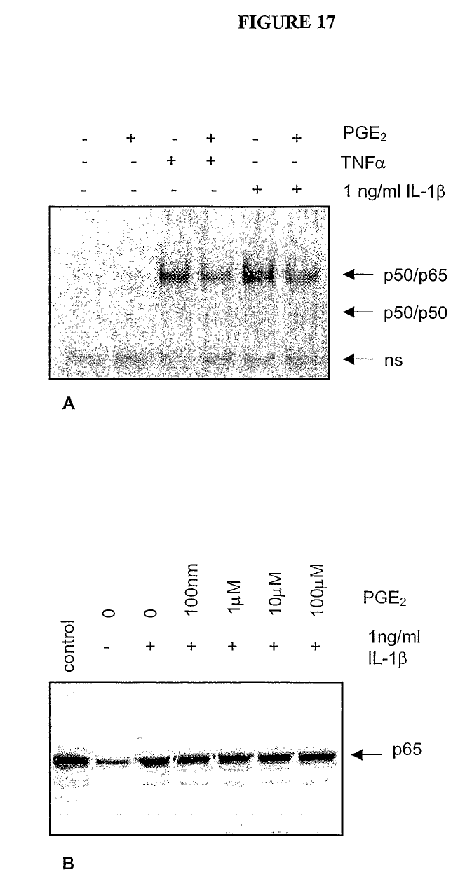

FIG. 17: PGE₂ does not inhibit TNFα- and IL-1β-induced NFκB activation

Analysis of nuclear protein extracts from myometrial cells treated with PGE₂ or vehicle for 2 h+/−TNFα or IL-1b stimulation (15 min). (A) EMSA using consensus κB probe. (B) Western immunoblot probing for nuclear p65.

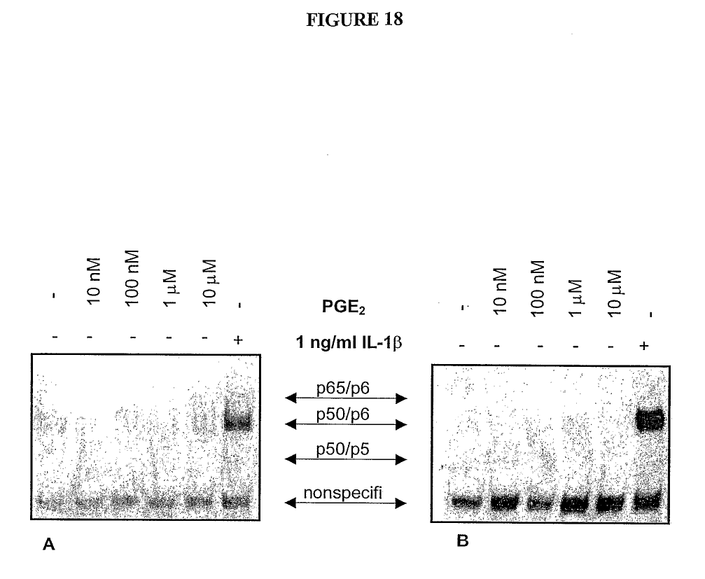

FIG. 18: PGE₂ does not induce NFκB DNA binding

EMSA analysis of nuclear protein extracts from (A) L− amnion cells and (B) myometrial cells treated with vehicle, PGE₂ or IL-1β. Consensus κB probe used.

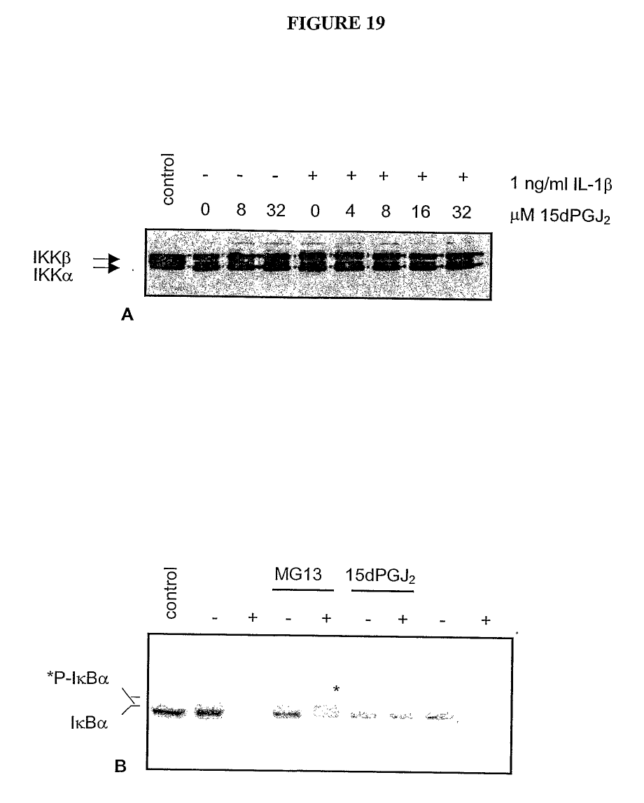

FIG. 19: 15dPGJ₂ inhibits IκBα phosphorylation

Western immunoblots of cytosolic extracts from myometrial cells (A) treated with 15dPGJ₂ for 2 h+/−IL1b for 15 min; probed for IKK, and (B) treated with 30 mM 15dPGJ₂, 40 mM MG132 or vehicle for 2 h, +/−IL-1β for 15 min; probed for IκBα.

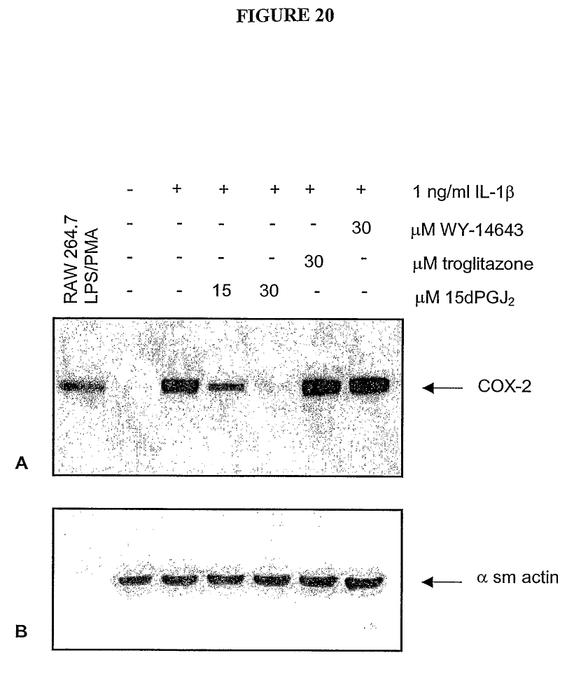

FIG. 20: Effect of 15dPGJ₂ and PPAR agonists on IL-1β-induced COX-2 protein expression

Western immunoblot of cytosolic protein extracts from myometrial cells treated with 15dPGJ₂, troglitazone, WY-14643 or vehicle for 2 h, followed by IL-1β stimulation for 6 h. Probed with antibodies to (A) COX-2, and (B) a smooth muscle actin.

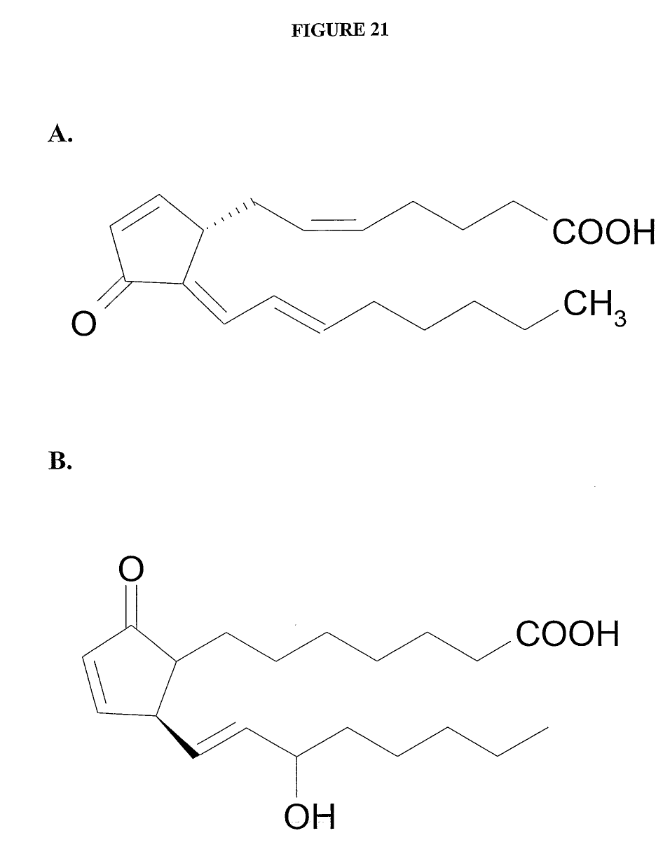

FIG. 21: Schematic of the structure of (A) prostaglandin A₁ (PGA₁) and (B) 15-deoxy-Δ^{12,14}prostaglandin J₂ (15-dPGJ₂)

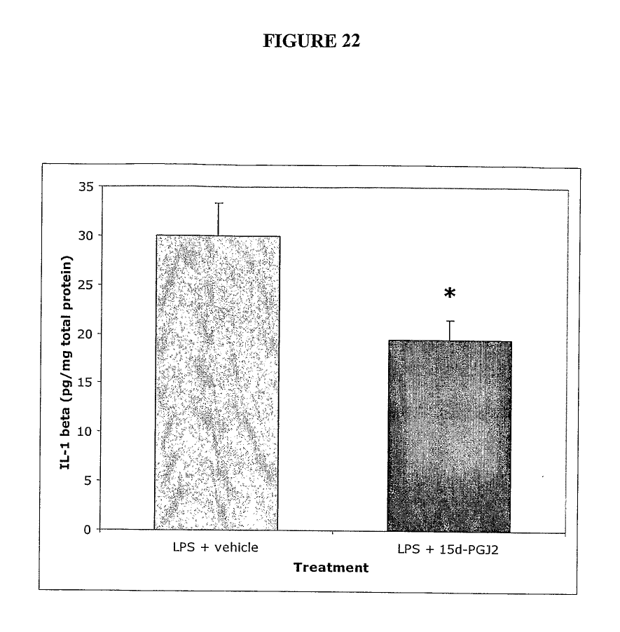

FIG. 22: Effect of LPS and 15d-PGJ₂ on inflammatory responses—IL-1β Levels

Concentrations of IL-1 (in placental homogenates collected from gestation day 16 mice 6 hours after intrauterine injection of 250 μg LPS+vehicle or 250 μg LPS+4 μg 15d-PGJ₂. * denotes statistically significant difference (t-test (p<0.05)).

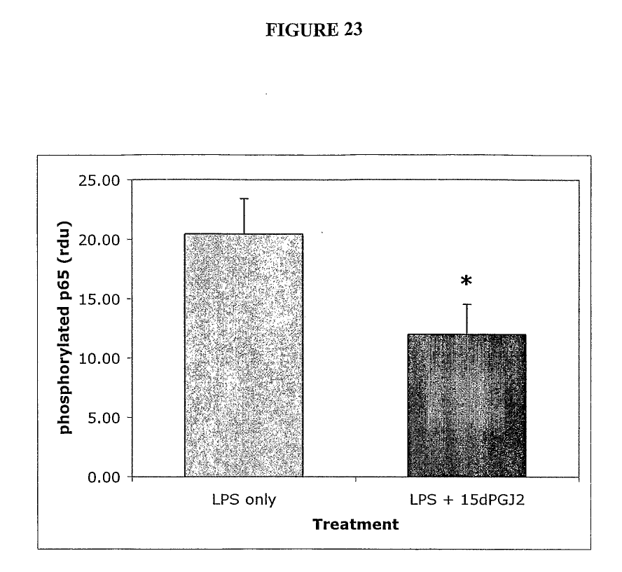

FIG. 23: Effect of LPS and 15d-PGJ₂ on inflammatory responses—phospho-p65 levels

Relative concentrations of phospho-p65 in placental homogenates collected from gestation day 16 mice 6 hours after intrauterine injection of 250 μg LPS+vehicle or 250 μg LPS+4 μg 15d-PGJ₂. * denotes statistically significant difference (t-test (p<0.05)).

### EXAMPLE 1

### Experimental Data

Methods

Abbreviations

EDTA Ethylenediaminetetraacetic acid

EGTA Ethyleneglycol bis-aminoethyltetra acetic acid

DTT Dithiotreitol

HEPES 4-(2-hydroxyethyl)-1-piperazineethanesulfonic acid

NP-40 Nonidet P-40

SDS-PAGE Sodium dodecyl sulphate—Polyacrylamide gel electrophoresis PVDF Polyvinylidene difluoride

PBS-T Phosphate Buffered Saline plus Tween

HRP Horseradish peroxidase

PBS Phosphate Buffered Saline

FCS Foetal Calf Serum

DMEM Dulbecco's modified eagle's medium

Tissue Biopsies and Cell Culture

Local Ethics committee approval was obtained for the collection of these tissues and patients gave informed consent.

Human Myometrial Cell Culture

Myometrial tissue was collected at term from the upper margin of uterine incision at the time of lower segment caesarean section either prior to the onset of labour (L−) or during fetal distress (L+). L+ samples were collected by Dr Mark Johnson and Dr S Soorrana at Chelsea & Westminster Hospital. Myometrial tissue was dissected, rinsed in PBS, and digested in serum-free DMEM containing 15 mg/ml collagenase 1A (Sigma), 15 mg/ml collagenase X, and 50 mg/ml bovine serum albumin for 45 min at 37° C. The cell suspension was filtered through a cell strainer, centrifuged at 400 g for 5 min, and the pellet re-suspended and plated out in DMEM, 10% FCS (Helena BioScience), 1% L-glutamine, 1% penicillin-streptomycin. Cells were used between passage numbers 1-4.

Human Amnion Cell Culture

Placentae were obtained from patients at term either at elective Caesarean section prior to labour (L−) or following spontaneous labour onset and vaginal delivery (L+). Amnion cells were prepared as described in Bennett et al., (1989). Briefly, the amnion was separated from the placenta, washed 3× in PBS, cut into strips, and incubated in 0.51 mM EDTA in PBS for 15 min. The strips were washed in PBS 2× and digested with 2.5 mg/ml dispase in serum-free DMEM for 35 min at 37° C. The amnion was then shaken vigorously in DMEM, 10% FCS to dissociate the cells, the remaining strips discarded, and the cell suspension pelleted at 175 g for 10 min and cultured in DMEM, 10% FCS (Sigma), 1% L-glutamine, 1% penicillin-streptomycin.

Protein Extracts from Cultured Cells

Nuclear and cytosolic protein extracts were obtained from cultured amnion cells as described by Schreiber et al (1989). For nuclear/cytosolic fractionation, confluent cell monolayers were scraped and lysed using a buffer containing 10 mM HEPES, 10 mM KCl, 0.1 mM EDTA, 0.1 mM EGTA, 2 mM DTT, 1% (v/v) NP-40 and complete protease inhibitor tablets (CPIs, Roche), diluted to manufacturer's instructions. Cell lysates were incubated on ice for 10 min and NP-40 added to a final concentration of 1% (v/v). Lysates were vortexed for 10 secs and centrifuged for 30 secs at 4° C., 12000 g. The supernatants were retained as the cytosolic protein extracts. The pellets were resuspended in buffer containing 10 mM HEPES, 10 mM KCl, 0.1 mM EDTA, 0.1 mM EGTA, 2 mM DTT, 400 mM NaCl, 1% NP-40 (v/v) and CPIs. Samples were shaken vigorously for 15 min in an ice bath. The nuclear protein extracts were obtained in the supernatant following a 5 min centrifugation at 4° C., 12000 g.

For whole cell lysates, confluent cell monolayers were scraped and lysed in a high-salt extraction buffer containing 0.4M KCl, 20 mM HEPES, 20% (v/v) glycerol, 1 mM DTT, and CPIs.

Protein Extracts from Fresh Tissue Biopsies

Tissue samples were rinsed in ice-cold PBS, dissected, flattened between aluminium foil, flash-frozen in liquid nitrogen, and stored at −80° C. Samples were reduced to powder in liquid nitrogen using a pestle and mortar. Powdered tissue was homogenized in a Dounce homogeniser on ice in a buffer containing 0.6% (v/v) NP-40, 150 mM HEPES, 1 mM EDTA, 0.5 mM PMSF and any unbroken tissue was removed by centrifugation for 30 sec at 200 rpm at 0° C. The supernatant was incubated on ice for 5 min, centrifuged for 10 min at 4000 rpm at 0° C., and the nuclear pellets resuspended in 25% (v/v) glycerol 20 mM HEPES, 0.42M NaCl, 1.2 mM MgCl₂, 0.2 mM EDTA, 0.5 mM DTT, and CPIs.

All extracts were aliquoted, frozen on dry ice and stored at −80° C. The extracts were processed for protein quantitation by the Lowry method using Bio-Rad protein assay reagents (Bio-Rad Laboratories) according to manufacturer's instructions.

Electro-Mobility Shift Assay (EMSA)

Oligonucleotide Labelling

Sense and antisense strands (175 nmole/ml each) were incubated in annealing buffer (10 mM Tris-HCl pH7.5, 100 mM NaCl, 1 mM EDTA) for 10 min at 65° C., and allowed to cool at room temperature for 2 h. 3.5 pmole double-stranded oligonucleotides were end-labelled with 0.37 MBq ³²P(γATP) by incubating for 30 min at 37° C. with T4 polynucleotide kinase. Labelled oligonucleotides were recovered by centrifugation at 300 rpm for 2 min through MicroSpin G-25 or G-50 sephadex columns (Amersham Biosciences).

EMSA

3-5 μg protein extracts were incubated on ice for 1 h with non-radiolabelled non-specific oligonucleotide (poly(dI-dC) or Oct-1) in a binding buffer (20% (v/V) glycerol, 5 mM MgCl₂, 2 mM EDTA, 50 mM Tris-HCl pH7.5, 250 nM NaCl, 2 mM DTT), followed by a 45 min incubation with 0.035 pmole ³²P(γATP)-end labelled oligonucleotide probes:

consensus NF-κB:
5′-AGT TGA GGG GAC TTT CCC AGG C-3′
consensus Oct-1:
5′-TGT CGA ATG CAA ATC ACT AGA A-3′
consensus SP-1:
5′-ATT CGA TCG GGG CGG GGC GAG
upstream COX-2 κB:
5′-CGG GAG AGG GGA TTC CCT GCG C-3′
downstream COX-2 κB:
5′-AGA GTG GGG ACT ACC CCC TCT-3′

Oct-1 or SP-1 consensus sequences were used as a controls for a NF-κB-specific effect. The resulting protein/DNA complexes were separated in a 4% acrylamide gel, the gel dried under vacuum at 80° C. and exposed to X-ray film. For supershift analysis, samples were incubated with 2 μg antibodies for 30 min on ice prior to incubation with oligonucleotides. Non-radio-labelled oligonucleotides were used at 100-fold molar excess for specific and non-specific competition for DNA binding. Reagents for EMSA were obtained from Promega Life Sciences, Delta House, Chilworth Research Centre, Southampton SO16 7NS, United Kingdom.

SDS-PAGE and Western Blotting Analysis

Protein samples (20-70 μg) were mixed with Laemmli sample buffer (1:1) containing β-mercaptoethanol (5%), and boiled for 5 min. Proteins were then separated by SDS-PAGE (12-14% gels) and transferred onto PVDF membrane (Amersham Pharmacia Biotech). The membranes were blocked overnight in 5% non-fat milk prepared in PBS-T buffer, at 4° C. The blots were incubated with the primary antibody in 1% non-fat milk in PBS-T buffer for 1 h, and washed three times (10 min each) in PBS-T with vigorous shaking. The blots were then incubated with HRP-conjugated secondary antibody (diluted 1:2000 in 1% non-fat milk in PBS-T buffer) for 1 h and washed three times (10 min each) in PBS-T. Signal detection was achieved using enhanced chemi-luminescence (ECL plus system, Amersham Pharmacia Biotech) according to manufacturer's instructions.

To re-probe a membrane, blots were incubated for 30 min in 50° C. stripping buffer (2% SDS, 62.5mM Tris-HCl pH6.7, 100 mM 2-mercaptoethanol), washed 2× in PBS-T, placed in blotto overnight, and then probed with a new antibody as above.

30-50 μg protein extracts were subjected to SDS-PAGE and Western immuno-blotting. Secondary antibodies were IgG-HRP and ECL Plus detection kit (Amersham Pharmacia Biotech, Amersham Place, Little Chalfont, Bucks, HP7 9NA) was used for visualisation.

Transfections and Luciferase Assay

Cells at 70-80% confluence in 24-well plates were transfected using the liposome Transfast (Promega). 0.5 μg per well of luciferase reporter construct was transfected using a 1:1 ratio of transfection (i.e., 3 μl Transfast per 1 μg DNA) in serum-free DMEM for 1 h. DMEM, 10% FCS was then added and the cells were incubated at 37° C. for 24 h. The medium was replaced with DMEM, 2% FCS for a further 24 h, and the cells treated with various agonists/inhibitors or vehicle for 6-8 h. Transfections were analysed in a dual firefly/renilla (Packard BioSciences/Calbiochem) luciferase assay or firefly/β-galactosidase (Promega/Galacton) assay using a luminometer.

pGL3.6 κB.BG.luc was the reporter construct used to assess NF-κB-mediated transcription, while the mutant pGL3.6 κBmut.luc and empty pGL3.BG.luc were used as controls (Schwarzer et al., 1998).

pGL3.6 κB.BG.luc: a NF-κB-dependent reporter construct with 6 copies of the NF-κB binding site. It contains two tandem repeats of the sequence 5′-GGG GAC TTT C CC TGG GGA CTT TCC CTG GGG ACT TTC CC-3′, which contains three copies of the decameric NF-κB binding site (underlined) upstream of a minimal β-globin promoter driving a luciferase gene.

pGL3.6 κBmut.luc: this reporter construct is as above except that the core NF-κB binding site is mutated to 5′-GCC ACT TTC C-3′ (mutated bases underlined).

pGL3.BG.luc: this reporter construct contains only the minimal β-globin promoter.

Cells were co-transfected with the renilla vector pRL-CMV or a β-galactosidase vector pCH110 as internal controls for transfection efficiencies.

In Vitro Translation and Plasmid Preps

For recombinant production of p65, a pSG5/p65 expression construct was transcribed and translated using a TNT Coupled Reticulocyte Lysate System (Promega), according to manufacturer's instructions. QIAGEN Maxi Prep kits were used for plasmid isolation from transformed JM109 E. coli cells, and all constructs were subsequently precipitated with polyethylene glycol.

Reagents/Antibodies

Recombinant cytokine IL-1 and TNFα from R&D Systems; 15d-PGJ₂, PGA₁, troglitazone, GW-9662, and 16,16-dimethyl-PGE₂ from Cayman Chemical; WY-14643, MG132 proteasome inhibitor, and PG490 (triptolide) from Calbiochem; HRP-conjugated secondary antibodies and antibodies to p50, p65, c-rel, RelB, COX-2, IκBα, IκBβ, and PPARγ from Santa Cruz; antibodies to p52, Bcl-3 and smooth muscle actin from Upstate Biotechnologies. Antibody to PPAR-γ from Affinity BioReagents, to phospho-p65 from Cell Signaling, to COX-1 from Alexis Biochemicals, and to lamin B from Oncogene Research Products.

Mouse Model of Preterm Labour

Surgery was performed on timed pregnant MF1 mice at day 16 of gestation. After deep maternal anaesthesia was attained, a minilaparotomy was performed in the lower abdomen. The uterine horns were exposed through the incision and preterm labour was induced by the intrauterine injection of 250 g lipopolysaccharide (LPS, Sigma) into the gravid horn. This was immediately followed by injection of 4 μg 5d-PGJ₂ (Cayman), or an equal volume of vehicle (methyl acetate), at the same site. The uterus was then returned to the abdomen and the fascia and skin were closed with continuous vicryl sutures.

Effect of LPS and 15d-PGJ₂ on Inflammatory Responses

Mice were sacrificed 6 hours after injection of LPS±15d-PGJ2. Placentae were washed in phosphate buffered saline (PBS), flash frozen in liquid nitrogen and stored at −80° C. until further processing. Fetuses were washed in PBS, then immediately fixed in 4% paraformaldehyde for 24 h and then stored in 70% ethanol until further processing. Placentae were homogenized for 1 minute in the presence of lysis buffer comprising 400 mM KCl, 20 mM HEPES pH7.4, 1 mM dithiothreitol, 20% glycerol and 5% (v/v) protease inhibitor cocktail.

Homogenate levels of Interleukin-1β (IL-1β) and tumour necrosis factor α (TNFα) were determined in placental lysates by ELISA (R and D systems) according to manufacturers instructions. Total protein concentrations were determined for each homogenate and IL-1β and TNFα levels were expressed as pg/mg total protein. Homogenates were also subjected to polyacrylamide gel electrophoresis. Loading volumes were adjusted according to the protein content of each homogenate such that a constant amount of protein was run in each lane. Phosphorylated p65 (phospho-p65) was detected by western immunoblotting using a specific antibody (Santa Cruz) and quantified by densitometric analysis.

Results

CyPGs, But not PPAR Agonists, Inhibit NF-κB DNA Binding in Amnion and Myometrial Cells.

15d-PGJ₂ inhibited IL1-β-induced NF-κB DNA binding in a dose-dependent manner in myometrial cells, as well as in L− and L+ amnion cells (FIG. 1). Protein binding to a consensus Oct-1 or Sp-1 probe was unaffected by either IL-11 or 15d-PGJ₂ treatment, confirming that the effects observed are NF-κB-specific.

Since PPAR-γ is the putative endogenous receptor for 15d-PGJ₂, and PPAR expression may be affected by cytokines (Tontonoz et al., 1998, Tanaka et al., 1999), PPAR-γ protein expression was examined in myometrial and amnion cells. PPAR-γ was shown to be expressed predominantly in the nucleus of both cell types, and its expression was not affected by IL-11 or 15d-PGJ₂ treatment (FIG. 2). 15d-PGJ₂ can also transactivate PPAR-α, though more weakly than PPAR-γ (Forman et al., 1995). PPAR-α expression in myometrial and amnion cells was found to be predominantly cytoplasmic (FIG. 3).

The ability of synthetic PPAR agonists to mimic the inhibitory effects of 15d-PGJ₂ was examined. The PPAR-γ agonist troglitazone had no effect on NF-κB DNA binding at 10-50 μM doses, although it did cause a slight reduction at 100 μM (FIG. 4, 5). Troglitazone can transactivate PPAR-γ at 1 μM and induces weak interactions between PPAR-γ and the co-activators p300 and steroid receptor co-activator (SRC-1) at 10M doses; adipogenesis is positively regulated by PPAR-γ, and troglitazone can induce expression of adipogenic markers at 5-10 μM doses (Prusty et al., 2002). Thus, at 100 μM concentrations, it is unlikely that troglitazone is exerting a specific effect through PPAR-Y. Since structurally distinct PPAR ligands may differentially affect coactivator/corepressor recruitment, a new potent PPAR-γ agonist, which lacks the TZD moiety, was also used. This GW1929 ligand failed to inhibit NF-κB DNA binding (FIG. 6). The synthetic PPAR-α agonist WY-14643 can transactivate PPARα at 5-25 μM doses in a GAL4 chimera transfection system (Kehrer et al., 2001), but WY-14643 had no effect on NF-κB DNA binding, at 10-100 μM concentrations. To further investigate a potential role for PPAR-γ in mediating the inhibitory effects of 15d-PGJ₂, NF-κB DNA binding was assessed in cells treated with 15d-PGJ₂ in the presence of the selective PPAR-γ inhibitor GW-9662. GW9662 binds irreversibly to PPAR-γ through covalent modification of Cys²⁸⁵ in the ligand-binding domain (Leesnitzer et al., 2002). GW-9662 failed to alleviate 15d-PGJ₂ inhibition of NF-κB (FIG. 6).

In contrast, PGA₁, which does not act as a PPAR ligand but does contain a cyclopentenone ring, was able to inhibit NF-κB DNA binding in amnion and myometrial cells, albeit at much higher doses than 15d-PGJ₂ (FIG. 7).

CyPGs, But not PPAR Agonists, Inhibit NF-κB Transcriptional Activity.

To determine whether the cyPG effects on NF-κB DNA binding extend to inhibition of NF-κB transactivation potential, amnion cells were transfected with the NF-κB-dependent reporter κB.BG.Luc and treated with 15d-PGJ₂, PGA₁, troglitazone, WY-14643 or vehicle, followed by IL-1 stimulation (FIG. 8). Constitutive reporter activity was seen in both L− and L+ amnion cells, although the levels were lower and showed a greater increase with IL-1 [symbol] in L− cells, in agreement with previous studies by Allport et al (2001). Both 15d-PGJ₂ and PGA₁ inhibited IL-1-induced NF-κB transcriptional activity, whereas troglitazone and WY-14643 did not.

In myometrial cells, 15d-PGJ₂ inhibited IL-1-induced NF-κB transcriptional activity in a dose-dependent manner, reducing reporter activity to basal levels (FIG. 9). IL-1-induced NF-κB transcriptional activity was also reduced to basal levels by PGA₁, but not troglitazone, GW1929 or WY-14643 (FIG. 10, 11).

GW1929 and troglitazone were shown to be functional as PPAR-γ ligands, potentiating PPAR-γ-mediated transcription of a PPRE-dependent reporter in both cell types. Endogenous PPAR-γ levels were not sufficient to drive the PPRE reporter in the transfection system used, with transcription requiring overexpression of the receptor. Troglitazone was also unable to inhibit a NF-κB-dependent reporter in PPARγ-transfected cells, and PPARγ overexpression did not promote 15d-PGJ₂ inhibition of NF-κB transcriptional activity (FIG. 12, 13, 14).

CyPGs, But not PGE₂, Inhibit NF-κB Activation and IκB Degradation.

15d-PGJ₂ inhibited IL-1β-induced p65 nuclear translocation and p50 phosphorylation in myometrial cells and in L−, L+ amnion cells in a dose-dependent manner (FIG. 15). This was paralleled by inhibition of IL-1β-induced IκBα and IκBβ degradation. Similarly, PGA₁ inhibited p65 nuclear translocation and IκB [symbol] degradation in myometrial cells (FIG. 16). 16,16-Dimethyl-PGE₂, a PGE₂ analogue with increased half-life, did not inhibit NF-κB DNA binding (controlled for with Oct-1 binding) or IL-1β-induced p65 nuclear translocation in myometrial and amnion cells (FIG. 17). This is not unexpected, since, in contrast to the cyPGs, PGE₂ is known to be pro-inflammatory, does not contain a cyclopentenone ring, and does not activate PPAR-γ (Forman et al., 1995). 16,16-dimethyl-PGE₂ did not inhibit NF-κB DNA binding or p65 nuclear translocation in myometrial cells (FIG. 18). However, neither did it stimulate NF-κB activity as reported in T cells (Dumais et al., 1998), nor did it synergise with IL-1β or TNFα.

Effect of 15-dPGJ₂ on NF-κB Upstream Activators and Downstream Targets.

In contrast to the proteasome inhibitor MG132, which prevented IL-1β-induced IκBα degradation and resulted in the accumulation of undegraded, phosphorylated IκBα, accumulation of phosphorylated IκBα was not detected following 15-dPGJ₂ treatment, suggesting that 15-dPGJ₂ may be affecting IKKs or other upstream kinases (FIG. 19). Both IL-1β and 15-dPGJ₂ treatment had no effect on IKKα or IKKβ protein expression, although it is more likely that 15d-PGJ₂ would inhibit the kinase activity of the IKKs.

Since COX-2 is an important target gene for NF-κB in labour, the effect of 15-dPGJ₂ and PPAR agonists on COX-2 expression was assessed. IL-1β-induced COX-2, expression was inhibited by 15-dPGJ₂, but not by troglitazone or WY-14643 (FIG. 20). Similar results were obtained in L− and L+ amnion cells.

Effect of LPS on Pre-Term Delivery.

Pre-term delivery of pups occurred by 16 hours after injection of LPS using the mouse model of preterm labour, as set out in the methods above.

Effect of LPS and 15d-PGJ₂ on Inflammatory Responses

In all mice, levels of TNFα and IL-1β were significantly higher in the placentae proximal to the injection site compared to those in the opposite horn. Levels of IL-1β were approximately 40% lower in proximal placentae injected with LPS+15d-PGJ₂ compared to those given LPS+ vehicle (FIG. 22). This difference was statistically significant (p<0.05). In contrast, TNFα levels were not significantly altered according to drug treatment.

Significantly, placental levels of IL-1β were not altered according the proximity of the placenta to the site of injection, indicating that inflammatory response can be distributed throughout the uterus, irrespective of the site of infection. However, phospho-p65 levels were approximately 35% lower in proximal placentae injected with LPS+15d-PGJ₂ compared to those given LPS+ vehicle (FIG. 23) and this difference was statistically significant (p<0.05).

### CONCLUSIONS

NF-κB Inhibition by cyPGs

15d-PGJ₂ inhibited IL1-β-induced NF-κB DNA binding and NF-κB-mediated transactivation in myometrial cells, as well as in L− and L+ amnion cells. 15d-PGJ₂ inhibited the nuclear translocation and activation of NF-κB, at least in part, by preventing the degradation of IκB (by IL-1β.

In myometrial and amnion cells, which expressed both PPAR-α and PPAR-γ receptors, neither PPAR-γ nor PPAR-α agonists were able to inhibit IL-1β-induced NF-κB DNA binding or NF-κB transcriptional activity at doses shown to inhibit NF-κB in other cell types (Chinetti et al., 1998; Gupta et al., 2001), or even at higher concentrations. In a study investigating the potential functional interactions between PPAR-γ and NF-κB in adipocytes, PPAR-γ agonists did not impair TNFα-induced NF-κB activation, nuclear translocation, or DNA binding activity; rather, they antagonised the transcriptional regulatory activity of NF-κB, and PPAR-γ overexpression was required to demonstrate such inhibition (Ruan et al., 2003). In the present study, while PPAR-γ overexpression potentiated transactivation of a PPRE, it did not enable the PPAR-γ agonists to inhibit NF-κB transcription. In addition, 15d-PGJ₂ was able to inhibit NF-κB transcription in the absence of exogenous PPAR-γ and overexpression of this receptor did not promote inhibition.

IL-1-induced COX-2 expression was inhibited by 15d-PGJ₂ but not by PPAR agonists. While PPAR agonists are known to be anti-inflammatory and can inhibit COX-2 expression (Staels et al., 1998; Subbaramaiah et al., 2001), they have also been reported to enhance COX-2 expression in certain cell types (Meade et al., 1999; Ikawa et al., 2001; Pang et al., 2003).

CyPGs such as 15d-PGJ₂ are characterised by the presence of a cyclopentenone ring system containing an electrophilic carbon. This ring can react covalently with nucleophiles such as the free sulfhydryls of glutathione and cysteine residues in cellular proteins. Receptor-independent actions of 15d-PGJ₂ have been attributed to its cyclopentenone ring. NF-κB proteins contain a conserved cysteine residue in their DNA-binding domain (DBD) and alkylation of this cysteine impairs DNA binding (Toledano et al., 1993). In the present study, PGA₁, a cyPG that does not act as a PPAR-γ ligand, was able to inhibit NF-κB DNA binding and transactivation, albeit at higher concentrations than 15d-PGJ₂. This ability of PGA₁, but not PGE₂ or PPAR agonists, to mimic the effects of 15d-PGJ₂ suggests that these cyPGs may inhibit NF-κB in amnion and myometrial cells by virtue of their cyclopentenone ring. While direct modification of NF-κB cysteines has not been addressed in this study, both 15d-PGJ₂- and PGA₁-mediated inhibition of NF-κB was shown to involve the inhibition of IκBα degradation, suggesting that events further upstream in the NF-κB cascade are being targeted.

Thus, while PPAR activation may not be effectively anti-inflammatory in amnion and myometrium, the use of cyPGs should prove useful in repressing NF-κB, and therefore an array of pro-inflammatory and labour-associated genes, in these tissues. CyPG administration offers an attractive alternative approach to anti-inflammatory treatment since a potential specificity of cyPGs for IKKβ/IκBα would spare other potentially beneficial pathways of NF-κB activation (e.g., the processing of p105 and formation of p50 homodimers), which might be disrupted by more broad-spectrum NF-κB inhibitors. The use of the cyPGs, able to simultaneously trigger the inhibition of the pro-inflammatory NF-κB and harness the anti-inflammatory activities of endogenous cytoprotective molecules represents a novel therapeutic approach in the treatment of preterm labour and neurodevelopmental disorders of the neonate.

This study provides evidence that the mouse model used is an effective model for the study of preterm delivery and agents that may delay the onset of preterm delivery. The finding of lower levels of IL-1β and phospho-p65 in mice treated with the cyclopentenone prostaglandin 15d-PGJ₂ suggests that this compound is effective at blocking the inflammatory pathway induced by LPS treatment in vivo.

### EXAMPLE 2

### Preferred Pharmaceutical Formulations and Modes and Doses of Administration

The compounds of the present invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The compounds of the present invention can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for administration. A device which delivers a pulsed electric field to cells increases the permeability of the cell membranes to the drug, resulting in a significant enhancement of intracellular drug delivery.

Compounds can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of administration is the ReGel injectable system that is thermosensitive. Below body temperature, ReGel is an injectable liquid while at body temperature it immediately forms a gel reservoir that slowly erodes and dissolves into known, safe, biodegradable polymers. The active drug is delivered over time as the biopolymers dissolve.

The compounds of the invention can also be delivered orally. The process employs a natural process for oral uptake of vitamin B₁₂ in the body to co-deliver proteins and peptides. By riding the vitamin B₁₂ uptake system, the protein or peptide can move through the intestinal wall. Complexes are synthesised between vitamin B₁₂ analogues and the drug that retain both significant affinity for intrinsic factor (IF) in the vitamin B₁₂ portion of the complex and significant bioactivity of the drug portion of the complex.

Compounds can be introduced to cells by “Trojan peptides”. These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient (Derossi et al., 1998).

Preferably, the pharmaceutical formulation of the present invention is a unit dosage containing a daily dose or unit, daily sub-dose or an appropriate fraction thereof, of the active ingredient.

The compounds of the invention can be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

In human therapy, the compounds of the invention can be administered alone but will generally be administered in admixture with a suitable pharmaceutical excipient diluent or carrier selected with regard to the intended route of administration and standard pharmaceutical practice.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications. The compounds of the invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds of the invention can also be administered parenterally, for example, intravenously, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Generally, in humans, oral or parenteral administration of the compounds of the invention is the preferred route, being the most convenient.

For veterinary use, the compounds of the invention are administered as a suitably acceptable formulation in accordance with normal veterinary practice and the veterinary surgeon will determine the dosing regimen and route of administration which will be most appropriate for a particular animal.

The formulations of the pharmaceutical compositions of the invention may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Preferred unit dosage formulations are those containing a daily dose or unit, daily sub-dose or an appropriate fraction thereof, of an active ingredient.

A preferred delivery system of the invention may comprise a hydrogel impregnated with a compound of the invention, which is preferably carried on a tampon which can be inserted into the cervix and withdrawn once an appropriate cervical ripening or other desirable affect on the female reproductive system has been produced.

It should be understood that in addition to the ingredients particularly mentioned above the formulations of this invention may include other agents conventional in the art having regard to the type of formulation in question, for example those suitable for oral administration may include flavouring agents.

### EXAMPLE 3

### Exemplary Pharmaceutical Formulations

Whilst it is possible for a compound of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen-free.

The following examples illustrate pharmaceutical formulations according to the invention in which the active ingredient is a compound of the invention.

### EXAMPLE 3A

### Tablet

| | | |
|---|---|---|
| Active ingredient | 100 | mg |
| Lactose | 200 | mg |
| Starch | 50 | mg |
| Polyvinylpyrrolidone | 5 | mg |
| Magnesium stearate | 4 | mg |
| | 359 | mg |

Tablets are prepared from the foregoing ingredients by wet granulation followed by compression.

### EXAMPLE 3B

### Ophthalmic Solution

| | | |
|---|---|---|
| Active ingredient | 0.5 | g |
| Sodium chloride, analytical grade | 0.9 | g |
| Thiomersal | 0.001 | g |
| Purified water to | 100 | ml |
| pH adjusted to | 7.5 | |

### EXAMPLE 3C

### Tablet Formulations

The following formulations A and B are prepared by wet granulation of the ingredients with a solution of povidone, followed by addition of magnesium stearate and compression.

Formulation A

| | mg/tablet | mg/tablet |
|---|---|---|
| (a) Active ingredient | 250 | 250 |
| (b) Lactose B.P. | 210 | 26 |
| (c) Povidone B.P. | 15 | 9 |
| (d) Sodium Starch Glycolate | 20 | 12 |
| (e) Magnesium Stearate | 5 | 3 |
| | 500 | 300 |

Formulation B

| | mg/tablet | mg/tablet |
|---|---|---|
| (a) Active ingredient | 250 | 250 |
| (b) Lactose | 150 | — |
| (c) Avicel PH 101 ® | 60 | 26 |
| (d) Povidone B.P. | 15 | 9 |
| (e) Sodium Starch Glycolate | 20 | 12 |
| (f) Magnesium Stearate | 5 | 3 |
| | 500 | 300 |

Formulation C

| | mg/tablet |
|---|---|
| Active ingredient | 100 |
| Lactose | 200 |
| Starch | 50 |
| Povidone | 5 |
| Magnesium stearate | 4 |
| | 359 |

The following formulations, D and E, are prepared by direct compression of the admixed ingredients. The lactose used in formulation E is of the direction compression type.

Formulation D

| | mg/capsule |
|---|---|
| Active Ingredient | 250 |
| Pregelatinised Starch NF15 | 150 |
| | 400 |

Formulation E

| | mg/capsule |
|---|---|
| Active Ingredient | 250 |
| Lactose | 150 |
| Avicel ® | 100 |
| | 500 |

Formulation F (Controlled Release Formulation)

The formulation is prepared by wet granulation of the ingredients (below) with a solution of povidone followed by the addition of magnesium stearate and compression.

| | mg/tablet |
|---|---|
| Active Ingredient | 500 |
| Hydroxypropylmethylcellulose | 112 |
| (Methocel K4M Premium) ® | |
| Lactose B.P. | 53 |
| Povidone B.P.C. | 28 |
| Magnesium Stearate | 7 |
| | 700 |

Drug release takes place over a period of about 6-8 hours and was complete after 12 hours.

### EXAMPLE 3D

### Capsule Formulations

Formulation A

A capsule formulation is prepared by admixing the ingredients of Formulation D in Example C above and filling into a two-part hard gelatin capsule. Formulation B (infra) is prepared in a similar manner.

Formulation B

| | mg/capsule |
|---|---|
| Active ingredient | 250 |
| Lactose B.P. | 143 |
| Sodium Starch Glycolate | 25 |
| Magnesium Stearate | 2 |
| | 420 |

Formulation C

| | mg/capsule |
|---|---|
| Active ingredient | 250 |
| Macrogol 4000 BP | 350 |
| | 600 |

Capsules are prepared by melting the Macrogol 4000 BP, dispersing the active ingredient in the melt and filling the melt into a two-part hard gelatin capsule.

Formulation D

| | mg/capsule |
|---|---|
| Active ingredient | 250 |
| Lecithin | 100 |
| Arachis Oil | 100 |
| | 450 |

Capsules are prepared by dispersing the active ingredient in the lecithin and arachis oil and filling the dispersion into soft, elastic gelatin capsules.

Formulation E (Controlled Release Capsule)

The following controlled release capsule formulation is prepared by extruding ingredients a, b, and c using an extruder, followed by spheronisation of the extrudate and drying. The dried pellets are then coated with release-controlling membrane (d) and filled into a two-piece, hard gelatin capsule.

| | mg/capsule |
|---|---|
| Active ingredient | 250 |
| Microcrystalline Cellulose | 125 |
| Lactose BP | 125 |
| Ethyl Cellulose | 13 |
| | 513 |

### EXAMPLE 3E

### Injectable Formulation

| | | |
|---|---|---|
| Active ingredient | 0.200 | g |
| Sterile, pyrogen free phosphate buffer (pH 7.0) to | 10 | ml |

The active ingredient is dissolved in most of the phosphate buffer (35-40° C.), then made up to volume and filtered through a sterile micropore filter into a sterile 10 ml amber glass vial (type 1) and sealed with sterile closures and overseals.

### EXAMPLE 3F

### Intramuscular Injection

| | |
|---|---|
| Active ingredient | 0.20 g |
| Benzyl Alcohol | 0.10 g |
| Glucofurol 75 ® | 1.45 g |
| Water for Injection q.s. to | 3.00 ml |

The active ingredient is dissolved in the glycofurol. The benzyl alcohol is then added and dissolved, and water added to 3 ml. The mixture is then filtered through a sterile micropore filter and sealed in sterile 3 ml glass vials (type 1).

### EXAMPLE 3G

### Syrup Suspension

| | |
|---|---|
| Active ingredient | 0.2500 g |
| Sorbitol Solution | 1.5000 g |
| Glycerol | 2.0000 g |
| Dispersible Cellulose | 0.0750 g |
| Sodium Benzoate | 0.0050 g |
| Flavour, Peach 17.42.3169 | 0.0125 ml |
| Purified Water q.s. to | 5.0000 ml |

The sodium benzoate is dissolved in a portion of the purified water and the sorbitol solution added. The active ingredient is added and dispersed. In the glycerol is dispersed the thickener (dispersible cellulose). The two dispersions are mixed and made up to the required volume with the purified water. Further thickening is achieved as required by extra shearing of the suspension.

### EXAMPLE 3H

### Suppository

| | mg/suppository |
|---|---|
| Active ingredient (63 μm)* | 250 |
| Hard Fat, BP (Witepsol H15 - Dynamit Nobel) | 1770 |
| | 2020 |

*The active ingredient is used as a powder wherein at least 90% of the particles are of 63 μm diameter or less.

One fifth of the Witepsol H15 is melted in a steam-jacketed pan at 45° C. maximum. The active ingredient is sifted through a 200 μm sieve and added to the molten base with mixing, using a silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 μm stainless steel screen and, with continuous stirring, is allowed to cool to 40° C. At a temperature of 38° C. to 40° C. 2.02 g of the mixture is filled into suitable plastic moulds. The suppositories are allowed to cool to room temperature.

### EXAMPLE 31

### Pessaries

| | mg/pessary |
|---|---|
| Active ingredient | 250 |
| Anhydrate Dextrose | 380 |
| Potato Starch | 363 |
| Magnesium Stearate | 7 |
| | 1000 |

The above ingredients are mixed directly and pessaries prepared by direct compression of the resulting mixture.

### EXAMPLE 3J

### Creams and Ointments

Described in Remington.

### EXAMPLE 3K

### Microsphere Formulations

The compounds of the invention may also be delivered using microsphere formulations, such as those described in Cleland (1997; 2001).

### EXAMPLE 3L

### Dry Powder Inhalation

The compounds of the invention may be delivered by inhalation, with the aid of a dry powder inhaler delivering micronised particles in metered quantities as described in Ansel (1999).

### EXAMPLE 3M

### Aerosol Inhalation

The compounds of the invention may be delivered by inhalation, with the aid of a suitable inhaler delivering micronised particles in metered quantities employing a non CFC propellant as described in Ansel (1999).

### REFERENCES

Allport V C, Pieber D, Slater D M, Newton R, White J O, Bennett P R. Human labour is associated with nuclear factor-κB activity which mediates cyclo-oxygenase-2 expression and is involved with the functional progesterone withdrawal, Mol Hum Reprod 2001; 7: 581-586.
Ansel. Pharmaceutical Dosage Forms and Drug Delivery Systems, 1999, Lippincott Williams and Wilkins.
Bendixen A C, Shevde N K, Dienger K M, Willson T M, Funk C D, Pike J W. IL-4 inhibits osteoclast formation through a direct action on osteoclast precursors via peroxisome proliferator-activated receptor γ1. Proc Natl Acad Sci USA 2001; 98: 2443-2448.
Bennett P R, Rose M P, Myatt L, Elder M G. Preterm labor: stimulation of arachidonic acid metabolism in human amnion cells by bacterial products. Am J Obstet Gynecol. 1987, 156:649-5.
Bennett, P. and Slater, D. The role of cyclo-oxygenases in the onset of labour., in improved non-steroid anti-inflammatory drugs: COX-2 enzyme inhibitors. J. Vane, R. Botting, and G. Botting, Editors. 1996, Kluwer Academic: London. P. 112-118.
Brown, N. L., Alvi, S. A., Elder, M. G., Bennett, P. R. and Sullivan, M. H. A spontaneous induction of fetal membrane prostaglandin production precedes clinical labour. J Endocrinol 1998; 157: R1-R6.
Carlon et al., Obstet Gynecol, 1992. 85(5), 769-774.
Chinetti G, Griglio S, Antonucci M, Torra I P, Delerive P, Majd Z, Fruchart J-C, Chapman J, Najib J, Staels B. Activation of proliferator-activated receptors α and γ induces apopotosis of human monocyte-derived macrophages. J Biol Chem 1998; 273: 25573-25580.
Cleland. Pharm. Biotechnol. 10:1-43,
Cleland et al. (2001) J. Control. Release 72:13-24 and
Corey, E. J. The logic of chemical synthesis-mulitstep synthesis of complex carbogenic molecules. Angew Chem Int Ed Engl, 1991, 30:455-465.
Crankshaw, D. J. and Dyal, R. Effects of some naturally occurring prostanoids and some cyclo-oxygenase inhibitors on the contractility of the human lower uterine segment in vitro. Can J Physiol Pharmacol, 1994. 72(8): p. 870-4.

Dammann O and Leviton A. Brain damage in preterm newborns: Might enhancement of developmentally regulated endogenous protection open a door for prevention? Pediatrics 1999; 104: 541-550.
Derossi et al. (1998), Trends Cell Biol 8, 84-87.
Dignam et al., Accurate transcription initiation by RNA polymerase II in a soluble extract from isolated mammalian nuclei. Nucleic Acids Res., 11:1475-1489.
Dyal R and Cranskshaw D J. The effects of some synthetic prostanoids on the contractility of the human lower uterine segment in vitro. Am J Obstet Gynecol 1988; 158: 281-285.
Elliot C L, Loudon J A, Brown N, Slater D M, Bennett P R, Sullivan M H. IL-1beta and IL-8 in human fetal membranes: changes with gestational age, labor, and culture conditions. Am J Reprod Immunol 2001; 46: 260-267.
Forman B M, Tontonoz P, Chen J, Brun R P, Spiegelman B M, Evans R M. 15-deoxy-Δ^{12,14}-prostaglandin J₂ is a ligand for the adipocyte determination factor PPARγ. Cell 1995; 83: 803-812.
Ganstrom et al., Acta Obstet Gynecol Scand, 1987. 66:429-431.
Garfield R E and Hertzberg E L. Cell-to-cell coupling in the myometrium: Emil Bozler's prediction. Prog Clin Biol Res 1990; 327: 673-681.
Gibson C S, MacLennen A H, Goldwater P N, Dekker G A. Antenatal causes of cerebral palsy: associations between inherited thrombophilias, viral and bacterial infection, and inherited susceptibility to infection. Obstet Gynecol Survey 2003; 58: 209-220.
Gupta R A, Polk D B, Krishna U, Israel D A, Yan F, DuBois R N, Peek R M Jr. Activation of peroxisome proliferator-activated receptor γ suppresses-nuclear factor κB-mediated apoptosis induced by Helicobacter Pylori I gastric epithelial cells. J Biol Chem 2001; 276: 31059-31066.
Herman A, Groutzd A, Bukovsky I, Arieli S, Sherman D, Caspi E. A simplified pre-induction scoring method for the prediction of successful vaginal delivery based on multivariate analysis of pelvic and other obstetrical factors. J Perinat Med. 1993, 21:117-24.
Huang J T, Welch J S, Ricote M, Binder C J, Willson T M, Kelly C, Witztum J L, Funk C D, Conrad D, Glass C K. Interleukin-4-dependent production of PPAR-γ ligands in macrophages by 12/15-lipoxygenase. Nature 1999; 400: 378-382.
Ikawa H, Kameda H, Kamitani H, Baek S J, Nixon J B, His L C, Eling T E. Effect of PPAR activators on cytokine-stimulated cyclooxygenase-2 expression in human colorectal carcinoma cells. Exp Cell Res 2001; 267: 73-80.
Johnson C, Van Antwerp D, Hope T J. An N-terminal nuclear export signal is required for the nucleocytoplasmic shuttling of IκBα. EMBO J 1999; 23: 6682-6693.
Keelan J A, Marvin K W, Sato T A, Coleman M, McCowan L M, Mitchell M D. Cytokine abundance in placental tissues: evidence of inflammatory activation in gestational membranes with term and preterm parturition. Am J Obstet Gynecol 1999; 181: 1530-1536.
Keirse (1995) “Indomethacin tocolysis in pre-term labour” in Pregnancy and Childbirth Module (Eds. Enkin, M. W., Keirse, M. J. N. C., Renfrew, M. J., Neilson, J. P.) Cochrane Database of Systematic Reviews, No 04383, Oxford).
Kelly R W. Inflammatory mediators and cervical ripening. J Reprod Immunol 2002; 57: 217-224.
Kunsch C, Ruben S M, Rosen C A. Selection of optimal kappa B/Rel DNA-binding motifs: interaction of both subunits of NF-kappaB with DNA is required for transcriptional activation. Mol Cell Biol 1992; 12: 4412-4421.
Lee, Curr. Opin. Biotechnol., 2001, 11:81-84.
Lee Y, Allport V, Sykes A, Lindstrom T, Slater D, Bennett P. The effects of labour and of interleukin 1 beta upon the expression of nuclear factor kappa B related proteins in human amnion. Mol Hum Reprod 2003; 9: 213-8.
Leesnitzer L M, Parks D J, Bledsoe R K, Cobb J E, Collins J L, Consler T G, Davis R G, Hull-Ryde E A, Lenhard J M, Patel L, Plunket K D, Shenk J L, Stimmel J B, Therapontos C, Willson T M, Blanchard S G. Functional consequences of cysteine modification in the ligand binding sites of peroxisome proliferator activated receptors by GW9662. Biochem 2002; 41: 6640-50.
Li Q and Verma I M. NF-κB regulation in the immune system. Nature Reviews, 2002. 2:725-735.
Lin R, Gewert D, Hiscott J. Differential transcriptional activation in vitro by NF-κB/Rel proteins. J Biol Chem 1995; 270: 3123-3131.
Maul H, Nagel S, Welsch G, Schafer A, Winkler M, Rath W. Messenger ribonucleic acid levels of interleukin-1 beta, interleukin-6 and interleukin-8 in the lower uterine segment increased significantly at final cervical-dilatation during term parturition, while those of tumor necrosis factor alpha remained unchanged. Eur J Obstet Gynecol Reprod Biol 2002; 102:143-7.
Meade E A, McIntyre T M, Zimmerman G A, Prescott S M. Peroxisome proliferators enhance cyclooxygenase-2 expression in epithelial cells. J Biol Chem 1999; 274: 8328-8334.
Mitchell M D, Edwin S S, Lundin-Schiller S, Silver R M, Smotkin D, Trautman M S. Mechanism of interleukin-1 beta stimulation of human amnion prostaglandin biosynthesis: mediation via a novel inducible cyclooxygenase. Placenta 1993; 14: 615-625.
Miyahara T, Schrum L, Rippe R, Xiong S, Yee H F Jr, Motomura K, Anania F A, Willson T M, Tsukamoto H. Peroxisome proliferator-activated receptors and hepatic stellate cell activation. J Biol Chem 2000; 275: 35715-35722.
Moise et al. Effect of advancing gestational age on the frequency of fetal ductal constriction in association with maternal indomethacin use” Am. J. Obstet. Gynecol., 1995, 170(45), 1204-5.
Narumiya S. Structures, properties and distributions of prostanoid receptors. Adv Prost Thromb Leuk Res 1995; 23: 17-22.
Narumiya S, Ohno K, Fukushima M, Fujiwara M. Site and mechanism of growth inhibition by prostaglandins. III. Distribution and binding of prostaglandin A2 and delta 12-prostaglandin J2 in nuclei. J Pharmacol Exp Ther 1987; 242: 306-11.
Nasuhura et al., JBC, 1999. 274:19965.
Pang L, Nie M, Corbett L, Knox A J. Cyclooxygenase-2 expression by nonsteroidal anti-inflammatory drugs in human airway smooth muscle cells: Role of peroxisome proliferator-activated receptors. J Immunol 2003; 170: 1043-1051.
Phelps C B, Sengchanthalangsy L L, Malek S, Ghosh G. Mechanism of □B DNA binding by Rel/NF-κB dimers. J Biol Chem 2000; 275: 24392-24399.
Pieber D, Allport V C, Hills F, Johnson M, Bennett P R. Interactions between progesterone receptor isoforms in myometrial cells in human labour. Mol Hum Reprod. 2001, 7:875-9.
Prusty D, Park B-H, Davis K E, Farmer S R. Activation of MEK/ERK signaling promotes adipogenesis by enhancing peroxisome proliferators-activated receptor γ (PPARγ) and C/EBPα gene expression during the differentiation of 3T3-L1 preadipocytes. J Biol Chem 2002; 277: 46226-46232.
Rasanen and Jouppila. Fetal cardiac function and ductus arteriosus during indomethacin and sulindac therapy for threatened pre-term labour; A randomised study. Am J Obstet Gynecol 1995. 173(1), 20-25.
Remington: The Science and Practise of Pharmacy, 19^{th} ed., The Philadelphia College of Pharmacy and Science, ISBN 0-912734-04-3.
Respondek et al., Fetal echocardiography during indomethacin treatment. Ultrasound Obstet Gynecol, 1995. 5, 86-89.
Romero R, Parvizi S T, Oyarzun E, Mazor M, Wu Y K, Avila C, Athanassiadis A P, Mitchell M D. Amniotic fluid interleukin-1 in spontaneous labour at term. J Reprod Med 1990; 35: 235-238.
Romero R, Espinoza J, Chaiworapongsa T, Kalache K. Infection and prematurity and the role of preventive strategies. Semin Neonatol. 2002; 7:259-74.
Ruan H, Pownall H J, Lodish H F. Troglitazone antagonizes TNF-α-induced reprogramming of adipocyte gene expression by inhibiting the transcriptional regulatory functions of NF-κB. J Biol Chem 2003; Manuscript M303141200.
Rush R W, Keirse M J N C, Howat P, Baum J D, Anderson A B, Turnbull A C. Contribution of preterm delivery to perinatal mortality. Br Med J 1976; 2: 965.
Satoh K, Yasumizu T, Fukuoka H, Kinoshita K, Kaneko Y, Tsuchiya M, Sakamoto S. Prostaglandin F2 alpha metabolite levels in plasma, amniotic fluid, and urine during pregnancy and labor. Am J Obstet Gynecol 1979; 133: 886-890.
Sambrook et al, Molecular Cloning. A laboratory manual. 1989. Cold Spring Harbour pub.
Schreiber et al., Rapid detection of octomer binding proteins with mini-extracts prepared form a small number of cells. Nucl. Acids Res, 1989. 17:6419.
Skinner K A and Challis J R. Changes in the synthesis and metabolism of prostaglandins by human fetal membranes and decidua at labour. Am J Obstet Gynecol 1985; 151: 519-523.
Slater D M, Berger L, Newton R, Moore G E, Bennett P R. Changes in the expression of types 1 and 2 cyclo-oxygenase in human fetal membranes at term. Am J Obstet Gynecol 1995; 172: 77-82.
Staels B, Koenig W, Habib A, Merval R, Lebret M, Torra I P, Delerive P, Fadel A, Chinetti G, Fruchart J C, Najib J, Maclouf J, Tedgui A. Activation of human aortic smooth-muscle cells is inhibited by PPARalpha but not by PPARgamma activators. Nature 1998; 393: 790-793.
Straus D. S. and Glass C. K. Cyclopentenone prostaglandins: new insights on biological activities and cellular targets. Med Res Rev, 2001. 21:185-210.

Subbaramaiah K, Lin D T, Hart J C, Dannenberg A J. Peroxisome proliferator-activated receptor γ ligands suppress the transcriptional activation of cyclooxygenase-2. J Biol Chem 2001; 276: 12440-12448.
Suyang H, Phillips R, Douglas I, Ghosh S. Role of unphosphorylated, newly synthesized IκBβ in persistent activation of NF-κB. Mol Cell Biol 1996; 16: 5444-5449.
Suzawa M, Takada I, Yanagisawa J, Ohtake F, Ogawa S, Yamauchi T, Kadowaki T, Takeuchi Y, Shibuya H, Gotoh Y, Matsumoto K, Kato S. Cytokines suppress adipogenesis and PPAR-γ function through the TAK1/TAB1/NIK cascade. Nature Cell Biol 2003; 5: 224-230.
Suzuki et al., Three component coupling synthesis of prostaglandins. A simplified, general procedure. Tetrahedron, 1990, 46:4809-4822.
Takata Y, Kitami Y, Yang Z-H, Nakamura M, Okura T, Hiwada K. Vascular inflammation is negatively autoregulated by interaction between CCAAT/enhancer-binding protein-δ and peroxisome proliferator-activated receptor-γ. Circ Res 2002; 91: 427-433.
Takeuchi et al. Adv. Drug. Delic. Rev., 2001, 47:39-54.
Tanaka T, Itoh H, Doi K, Fukunaga Y, Hosoda K, Shintani M, Yamashita J, Chun T H, Inoue M, Masatsugu K, Sawada N, Saito T, Inoue G, Nishimura H, Yoshimasa Y, Nakao K. Down regulation of peroxisome proliferator-activated receptor g expression by inflammatory cytokines and its reversal by thiazolidinediones. Diabetologia 1999; 42: 702-710.
Toledano M B, Ghosh D, Trinh F, Leonard W J. N-terminal DNA-binding domains contribute to differential DNA-binding specificities of NF-kappa B p50 and p65. Mol Cell Biol 1993; 13: 852-860.
Tontonoz P, Nagy L, Alvarez J, Thomazy V, Evans R. PPARγ promotes monocyte/macrophage differentiation and uptake of oxidized LDL. Cell 1998; 93: 241-252.
Tulzer et al., Doppler echocardiography of fetal ductus arteriosus constriction versus increased right ventricular output. JACC, 1991. 18(2), 532-36.
Turnbull, A. The fetus and birth, in Elsevier, London. 1977.
Urakubo A, Jarskog L F, Lieberman J A, Gilmore J H. Prenatal exposure to maternal infection alters cytokine expression in the placenta, amniotic fluid, and fetal brain. Schizophrenia research 2001; 47: 27-36.
Van Meir, C. A., et al., Chorionic prostaglandin catabolism is decreased in the lower uterine segment with term labour. Placenta, 1997. 18(2-3): p. 109-14.
Van Meir, C. A., et al., Immunoreactive 15-hydroxyprostaglandin dehydrogenase (PGDH) is reduced in fetal membranes from patients at pre-term delivery in the presence of infection. Placenta, 1996. 17(5-6): p. 291-7.
Volpe J J. Neurobiology of periventricular leukomalacia in the premature infant. Pediatr Res 2001; 50:553-62.
Ward C, Dransfield I, Murray J, Farrow S N, Haslett C, Rossi A G. Prostaglandin D₂ and its metabolites induce caspase-dependent granulocyte apoptosis that is mediated via inhibition of IκBα degradation using a peroxisome proliferator-activated receptor-γ-independent mechanism. J Immunol 2002; 168: 6232-6243.
Whiteside S T, Epinat J-C, Rice N R, Israel A. I kappa B epsilon, a novel member of the IκB family, controls RelA and cRel NF-κB activity. EMBO J 1997(b); 16: 1413-1426.
Zhou Y C and Waxman D J. Cross-talk between Janus-Kinase-signal transducer activator of transcription (JAK-STAT) and peroxisome proliferator-activated α (PPARα) signaling pathways. J Biol Chem 1999; 274: 2672-2681.

1. Use of a cyclopentenone prostaglandin in the manufacture of a medicament for delaying the onset and/or preventing the continuation of labour in a female.

2. Use of a cyclopentenone prostaglandin in the manufacture of a medicament for preventing and/or reducing an inflammatory response in the reproductive system of a female.

3. A use according to claim 2 wherein the female is pregnant.

4. A use according to claim 1 wherein the female is human and the duration of pregnancy is more than approximately 13 weeks.

5. A use according to claim 4 wherein the duration of pregnancy is approximately between 20 and 32 weeks.

6. A use according to claim 1 wherein the medicament reduces and/or prevents an inflammatory response in the reproductive system of a female associated with the onset or continuation of labour.

7. A use according to claim 1 wherein the medicament reduces and/or prevents an inflammatory response in the reproductive system of a female associated-with infection by a pathogenic agent.

8. A use according to claim 7 wherein the pathogenic agent is viral, bacterial or fungal.

9. A use according to claim 6 wherein the inflammatory response is activated by stretch of the uterus.

10. A use according to claim 1 wherein the medicament reduces and/or prevents one or more of the following conditions: pre-term labour; pathogenic infection; cervical ripening, uterine contractions.

11. A use according to claim 1 wherein the medicament reduces and/or prevents fetal or neonatal damage.

12. A use according to claim 11 wherein the fetal or neonatal damage is brain damage.

13. A use according to claim 12 wherein the fetal or neonatal damage is one or more of the following conditions: astrogliosis; loss of myelin-producing oligodendrocytes; multifocal necroses resulting in cystic change (periventricular leucomalacia, PVL).

14. A use according to claim 1 wherein the cyclopentenone prostaglandin is 15-deoxy-Δ^{12,14}-prostaglandin J₂ and/or prostaglandin A₁ and/or is a prodrug of 15-deoxy-Δ^{12,14}-prostaglandin J₂ and/or prostaglandin A₁.

15. A use according to claim 14 wherein the prodrug is PGD₂ or PGE₁.

16. A use according to claim 1 wherein the medicament further comprises a pharmaceutically acceptable excipient, diluent or carrier.

17. A use according to claim 1 wherein the medicament is in a form adapted for delivery by mouth.

18. A use according to claim 1 wherein the medicament is in a form adapted for delivery by intravenous injection.

19. A use according to claim 1 wherein the medicament is in a form adapted for delivery by intra-amniotic injection.

20. A use according to claim 1 wherein the medicament is in a form which is compatible with the amniotic fluid.

21. A use according to claim 1 wherein the medicament further comprises an agent for treating a female who has or is at risk of one or more of the following conditions: pre-term labour; pathogenic infection; cervical ripening, uterine contractions.

22. A use according to claim 21 wherein the agent is a corticosteroid.

23. A use according to claim 21 wherein the agent is capable of preventing and/or reducing respiratory distress syndrome in the neonate.

24. A use according to claim 23 wherein the agent is selected from dexamethasone or betamethasone.

25. A use according to claim 21 wherein the condition is preterm labour and the agent is capable of delaying delivery.

26. A use according to claim 21 wherein the condition is uterine contractions and the agent is a tocolytic agent.

27. A use according to claim 26 wherein the tocolytic agent is selected from oxytocin receptor antagonists, calcium channel blockers, sympathomimetics, nitric oxide donors.

28. A use according to claim 27 wherein the oxytocin receptor antagonist is Atosiban.

29. A use according to claim 27 wherein the calcium channel blocker is Nifedipine.

30. A use according to claim 27 wherein the sympathomimetic is Ritodrine.

31. A use according to claim 27 wherein the nitric oxide donor is glyceryl trinitrate.

32. A use according to claim 2 wherein the inflammatory response is mediated by NFκB in uterine cells.

33. A use according to claim 32 wherein the cyclopentenone prostaglandin is capable of inhibiting and/or reducing NFκB activity by preventing and/or reducing NFκB DNA-binding in uterine cells.

34. A use according to claim 33 wherein the cyclopentenone prostaglandin is capable of inhibiting and/or reducing NFκB activity by preventing and/or reducing NFκB-mediated transcriptional regulation in uterine cells.

35. A use according to claim 34 wherein the cyclopentenone prostaglandin is capable of inhibiting and/or reducing NFκB activity by preventing and/or reducing NFκB production in uterine cells.

36. A pharmaceutical composition comprising a cyclopentenone prostaglandin and a pharmaceutically acceptable carrier or excipient, the cyclopentenone prostaglandin being present in an amount effective to prevent and/or reduce an inflammatory response in the reproductive system of a female.

37. A method of treating inflammation within the reproductive system of a female, the method comprising administering an effective amount of a medicament as defined in claim 1 to a subject in need thereof.
